(19) United States
(12) Patent Application Publication
(10) Pub. No.: US 2009/0149485 A1
(43) Pub. Date: Jun. 11, 2009

(54) PYRIMIDINEDIONES AS TYROSINE KINASE INHIBITORS

(75) Inventors: Balaji Vituduki Narayana Iyengar, Bangalore (IN); Marahanakuli Dattatreya Prasanna, Bangalore (IN); Phukan Samiron, Bangalore (IN); Singh Sudhir Kumar, Bangalore (IN)

Correspondence Address: SUGHRUE MION, PLLC, 2100 PENNSYLVANIA AVENUE, N.W., SUITE 800, WASHINGTON, DC, 20037, US

(73) Assignee: Jubilant Biosys Limited, Bangalore (IN)

(21) Appl. No.: 12/257,962

(22) Filed: Oct. 24, 2008

Related U.S. Application Data
(63) Continuation-in-part of application No. PCT/IN2007/000157, filed on Apr. 23, 2007.

(30) Foreign Application Priority Data
Apr. 24, 2006 (IN) 738/CHE/2006

(51) Int. Cl. A61K 31/506; C07D 239/10; A61P 25/00; A61P 3/00

(52) U.S. Cl. 514/274; 544/310

(57) ABSTRACT

The present invention relates to a compound of formula (I)

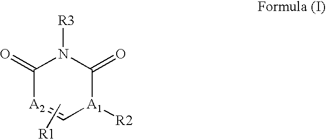

or its stereoisomers, tautomers, solvates, hydrates, prodrugs, pharmaceutically acceptable salts or mixtures thereof, or pharmaceutical composition comprising a compound of formula (I) or a pharmaceutically acceptable salt thereof. The present invention also provides a method for the prophylaxis or treatment of a medical condition associated with protein kinase, by administering a pharmaceutically effective amount of the compound of formula (I) or salts thereof.

This is a Continuation-in-Part of Application No. PCT/IN2007/000157 filed Apr. 23, 2007. The entire disclosure of the prior application, application number PCT/IN2007/000157 is considered part of the disclosure of the accompanying continuation-in-part application and is hereby incorporated by reference.

### TECHNICAL FIELD

The present invention relates to compound of formula (I) useful for inhibiting protein tyrosine kinases, compositions containing these compounds and a method of treatment thereof.

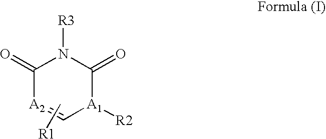

### BACKGROUND OF THE INVENTION

Protein kinases are the super family of proteins, which function as enzymes. They catalyze the transfer of the terminal or gamma phosphate group of ATP to acceptor target(s) protein at serine, threonine and tyrosine residues. These amino acid residues have hydroxyl group which is replaced by inorganic phosphate group during phosphorylation. Introduction of phosphate group on protein can i) create steric hindrance to alter protein binding, ii) can create or complete binding site for a complementary protein or iii) can cause a conformational change to alter protein activation causing a significant change in how the substrate protein interacts with other molecules. Phosphatases do the reverse of protein kinase by catalyzing the removal of the phosphate by hydrolysis of protein phosphate complex. Protein kinases and phosphatases play a pivotal role in regulating and coordinating aspects of metabolism, gene expression, cell growth, cell motility, cell differentiation, and cell division. Hence proper switching on and off protein kinases is crucial for orderly function of cell.

A key structural feature of the protein kinase family members is the catalytic domain or catalytic subunits, which is considered to be conserved throughout the protein kinase family and is approximately 250 amino acids long. There are other domains, which are involved in regulation, interactions with other partners or subcellular localization. These domains are called as regulatory domains or regulatory subunits. The regulatory domains serve two functions: (i) It targets the kinase to the appropriate cellular location and (ii) It regulates kinase activity by serving as an auto-inhibitory module.

The catalytic domain further constitutes 12 conserved sub domains i.e. sub-domain I-XII that fold into almost common catalytic core in all the protein kinases. For example, protein kinase A has 11 subunits in catalytic domain. Protein kinases show variability in other parts of kinase domains and different kinases may contain different domains, different subunits or both.

Three separate roles can be ascribed to catalytic domain of protein kinases:
i. Binding and orientation of ATP phosphate donor as a complex with divalent cation.
ii. Binding and orientation of the protein substrate.
iii. Transfer of the gamma-phosphate from ATP to the acceptor hydroxyl residue of the protein substrate.

Protein kinases are involved in signal transduction pathways linking growth factors, hormones and other call regulation molecules to cell growth, survival and metabolism under both normal and pathological conditions.

GSK-3 is a serine/threonine protein kinase comprised of isoforms that are each encoded by distinct genes. This enzymes participates in several signaling pathways important in disease and small molecule compounds are being developed as ATP competitive inhibitors. GSK3 inhibition by compounds may also promote proliferative disorders, e.g. colon cancer. GSK3 has been implicated in various diseases including diabetes, Alzheimer's disease, CNS disorders such as manic depressive disorder and neurodegenerative disease and cardiomyocyte hypertrophy [WO 2004/111008].

### Protein Tyrosine Kinases

Protein Tyrosine kinases (PTKs) are enzymes which catalyze the phosphorylation of specific tyrosine residues in cellular proteins. This post-translational modification of these substrate proteins, often enzymes themselves, acts as a molecular switch regulating cell proliferation, activation or differentiation (for review, see Schlessinger and Ulrich, 1992, Neuron 9:383-391). Aberrant or excessive PTK activity has been observed in many disease states including benign and malignant proliferative disorders as well as diseases resulting from inappropriate activation of the immune system (e.g., autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, infantile hemangiomas).

Tyrosine kinases can be of the receptor-type (having extracellular, transmembrane and intracellular domains) or the non-receptor type (being wholly intracellular).

### Glycogen Synthase Kinase-3 (GSK-3)

Glycogen synthase kinase-3 (GSK-3) is a serine/threonine protein kinase comprised of α and β isoforms that are each encoded by distinct genes [Coghlan et al., Chemistry & Biology, 7, 793-803 (2000); Kim and Kimmel, Curr. Opinion Genetics Dev., 10, 508-514 (2000)]. GSK-3 has been implicated in various diseases including diabetes, Alzheimer's disease, CNS disorders such as manic depressive disorder and neurodegenerative diseases, and cardiomyocete hypertrophy [WO 99/65897; WO 00/38675; and Haq et al., J. Cell Biol. (2000) 151, 117]. These diseases may be caused by, or result in, the abnormal operation of certain cell signaling pathways in which GSK-3 plays a role. GSK-3 has been found to phosphorylate and modulate the activity of a number of regulatory proteins. These proteins include glycogen synthase which is the rate limiting enzyme necessary for glycogen synthesis, the microtubule associated protein Tau, the gene transcription factor β-catenin, the translation initiation factor e1F2B, as well as ATP citrate lyase, axin, heat shock factor-1, c-Jun, c-Myc, c-Myb, CREB, and CEPB α. These diverse protein targets implicate GSK-3 in many aspects of cellular metabolism, proliferation, differentiation and development.

In a GSK-3 mediated pathway that is relevant for the treatment of type II diabetes, insulin-induced signaling leads to cellular glucose uptake and glycogen synthesis. Along this pathway, GSK-3 is a negative regulator of the insulin-induced signal. Normally, the presence of insulin causes inhibition of GSK-3 mediated phosphorylation and deactivation of glycogen synthase. The inhibition of GSK-3 leads to increased glycogen synthesis and glucose uptake [Klein et al., PNAS, 93, 8455-9 (1996); Cross et al., Biochem. J., 303, 21-26 (1994); Cohen, Biochem. Soc. Trans., 21, 555-567 (1993); Massillon et al., Biochem. J. 299, 123-128 (1994)]. However, in a diabetic patient where the insulin response is impaired, glycogen synthesis and glucose uptake fail to increase despite the presence of relatively high blood levels of insulin. This leads to abnormally high blood levels of glucose with acute and long term effects that may ultimately result in cardiovascular disease, renal failure and blindness. In such patients, the normal insulin-induced inhibition of GSK-3 fails to occur. It has also been reported that in patients with type II diabetes, GSK-3 is overexpressed [WO 00/386751]. Therapeutic inhibitors of GSK-3 therefore are considered to be useful for treating diabetic patients suffering from an impaired response to insulin.

GSK-3 activity has also been associated with Alzheimer's disease. This disease is characterized by the well-known β-amyloid peptide and the formation of intracellular neurofibrillary tangles. The neurofibrillary tangles contain hyperphosphorylated Tau protein where Tau is phosphorylated on abnormal sites. GSK-3 has been shown to phosphorylate these abnormal sites in cell and animal models. Furthermore, inhibition of GSK-3 has been shown to prevent hyperphosphorylation of Tau in cells [Lovestone et al., Current Biology 4, 1077-86 (1994); Brownlees et al., Neuroreport 8, 3251-55 (1997)]. Therefore, it is believed that GSK-3 activity may promote generation of the neurofibrillary tangles and the progression of Alzheimer's disease.

Another substrate of GSK-3 is β-catenin which is degradated after phosphorylation by GSK-3. Reduced levels of β-catenin have been reported in schizophrenic patients and have also been associated with other diseases related to increase in neuronal cell death [Zhong et al., Nature, 395, 698-702 (1998); Takashima et al., PNAS, 90, 7789-93 (1993); Pei et al., J. Neuropathol. Exp, 56, 70-78 (1997)].

As a result of the biological importance of GSK-3, there is current interest in therapeutically effective GSK-3 inhibitors. Small molecules that inhibit GSK-3 have recently been reported [WO 99/65897 (Chiron) and WO 00/38675 (SmithKline Beecham)].

For many of the aforementioned diseases associated with abnormal GSK-3 activity, other protein kinases have also been targeted for treating the same diseases. However, the various protein kinases often act through different biological pathways. For example, certain quinazoline derivatives have been reported recently as inhibitors of p³⁸ kinase (WO 00/12497 to Scios). The compounds are reported to be useful for treating conditions characterized by enhanced p³⁸-α activity and/or enhanced TGF-β activity. While p³⁸ activity has been implicated in a wide variety of diseases, including diabetes, p³⁸ kinase is not reported to be a constituent of insulin signaling pathway that regulates glycogen synthesis or glucose uptake. Therefore, unlike GSK-3, p³⁸ inhibition would not be expected to enhance glycogen synthesis and/or glucose uptake. U.S. Pat. No. 7,205,314 discloses isoquinoline derivative and treatment dementia related disease, Alzheimer's disease and conditions associated with glycogen syntheses kinase-3.

WO2006/055578 discloses the method for the asymmetric synthesis of (−)-agelastatin A for the treatment of disorders such as cancer, Alzheimer's disease, diabetes or stroke.

WO2005/061516 discloses a method for the treatment or prophylaxis of a disorder in a mammal, said disorder being characterized by misregulation of GSK-3, comprising, administering to the mammal a therapeutically effective amount of a fused pyrimidine compounds, or a physiologically functional derivative thereof. Preferably the disorder is Type II Diabetes.

WO2004/016612 relates to purine derivatives having antiproliferative properties which are useful in the treatment of proliferative disorders such as cancer, leukemia, psoriasis and the like. GSK3 inhibition is therefore of therapeutic significance in the treatment of diabetes, particularly type II, and diabetic neuropathy.

WO2004/013140 discloses pyrazole compounds for inhibiting GSK-3 activity and methods for treating or lessening the severity of diseases or conditions associated with GSK-3 in patients. The diseases or conditions amenable to the methods of this invention include, for example, neurological and neurodegenerative disorders, diabetes, psychiatric disorders, multiple sclerosis (MS), myocardial infarction, reperfusion/ischemia, baldness, and stroke.

The identification of effective small compounds which specifically inhibit signal transduction and cellular proliferation by modulating the activity of receptor and non-receptor tyrosine and serine/threonine kinases to regulate and modulate abnormal or inappropriate cell proliferation, differentiation, or metabolism is therefore desirable. In particular, the identification of methods and compounds that specifically inhibit the function of a tyrosine kinase which is essential for antiogenic processes or the formation of vascular hyperpermeability leading to edema, ascites, effusions, exudates, and macromolecular extravasation and matrix deposition as well as associated disorders would be beneficial.

### OBJECTS OF THE PRESENT INVENTION

Accordingly, the object of the present invention is to provide a compound of formula (I), its stereoisomers, tautomers, solvates, hydrates, prodrugs and salts, useful for inhibiting protein tyrosine kinases.

An object of the present invention is to provide a pharmaceutical composition comprising a compound of formula (I) or a pharmaceutically acceptable salt thereof, in combination with a pharmaceutically acceptable carrier or diluents.

Another object of the present invention is to provide a method for the prophylaxis or treatment of a medical condition associated with protein tyrosine kinase, by administering a pharmaceutically effective amount of the compound of formula (I) or a composition or salts thereof.

### SUMMARY OF THE INVENTION

The present invention provides a compound of formula (I)

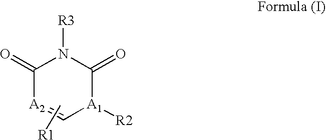

or its stereoisomers, tautomers, solvates, hydrates, prodrugs, pharmaceutically acceptable salts or mixtures thereof, wherein A₁ is nitrogen; A₂ is carbon;

R1 is selected from optionally substituted aryl, 5-6 membered heterocyclyl and heteroaryl with 1-3 hetero atoms independently selected from N, O, and S, wherein the substituents are selected from a group consisting of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said 5-6 membered heterocyclyl and heteroaryl exclude nucleosides, sugar moieties, oligomers, peptides, and polymeric ring systems;
R2 and R3 are independently selected from H, alkyl, —(CH₂)ₙ—Y wherein n=0 to 4, and Y is selected from a group consisting of alkyne, alkoxy, ester, carbonyl, carboxyl, aryl, cycloalkyl, heteroaryl and heterocyclyl which are further optionally substituted with halogen, cyano, nitro, alkyl, hydroxyl, alkoxy, carboxyl groups; with the proviso that either R2 or R3 is H and R2 is not same as R3.

The present invention also provides a pharmaceutical composition comprising a compound of formula (I) or a pharmaceutically acceptable salt thereof, in combination with a pharmaceutically acceptable carrier or diluents. The present invention also provides a method for the prophylaxis or treatment of a medical condition associated with protein kinase, by administering a pharmaceutically effective amount of the compound of formula (I) or salts thereof.

### DETAILED DESCRIPTION OF THE INVENTION

The list of abbreviations as used in the specification for the described expressions is as follows:

In the specification the connecting phrase “comprise” or “comprises” or “comprising” will be understood to imply the inclusion of a stated integer or groups of integers but not the exclusion of any other integer or groups of integers.

The following terms as used in the present specification have the meanings as indicated:

The term “aryl”, alone or in combination with other terms, refers to monocyclic or polycyclic aromatic carbon ring systems having five to fourteen members. Examples of aryl groups include, but are not limited to, phenyl (Ph), 1-naphthyl, 2-naphthyl, 1-anthracyl and 2-anthracyl.

The term “aralkyl” refers to an alkyl group substituted by an aryl. Also explicitly included within the scope of the term “aralkyl” are alkenyl or alkynyl groups substituted by an aryl. Examples of aralkyl groups include benzyl and phenethyl. The term “aryl”, “aryl group” or “aryl ring” also refers to rings that are optionally substituted, unless otherwise indicated.

The term “alkyl”, used alone or as part of a larger moiety, refers to both straight and branched saturated chains containing one to twelve carbon atoms.

The terms “alkenyl” and “alkynyl”, used alone or as part of a larger moiety, encompass both straight and branched chains containing two to twelve carbon atoms and at least one unit of unsaturation. An alkenyl group contains at least one carbon-carbon double bond and an alkynyl group contains at least one carbon-carbon triple bond.

The term “aliphatic” refers to straight chain or branched hydrocarbons that are completely saturated or that contain one or more units of unsaturation. For example, aliphatic groups include substituted or unsubstituted linear or branched alkyl, alkenyl and alkynyl groups. Unless indicated otherwise, the term “aliphatic” encompasses both substituted and unsubstituted hydrocarbons.

The term “ATP analogue” refers to a compound derived from Adenosine-5′-triphosphate (ATP). The analogue can be ADP, or non-hydralysable, for example, Adenylyl Imidodiphosphate (AMPPNP). AMPPNP can be in complex with Magnesium or Manganese ions.

The term “binding pocket” refers to a region of a molecule or molecular complex, that, as a result of its shape, favorably associates with another chemical entity or compound.

The term “biological sample” includes, without limitation, cell cultures or extracts thereof; biopsied material obtained from a mammal or extracts thereof; and blood, saliva, urine, faeces, semen, tears, or other body fluids or extracts thereof.

The term “carbocylyl” or “carbocyclic”, alone or in combination with any other term, refers to monocyclic or polycyclic non-aromatic carbon ring systems, which may contain a specified number of carbon atoms, preferably from 3 to 12 carbon atoms, which are completely saturated or which contain one or more units of unsaturation. A carbocyclic ring system may be monocyclic, bicyclic or tricyclic. A carbocylyl ring may be fused to another ring, such as an aryl ring or another carbocyclic ring. Examples of carbocyclic rings could include cyclohexyl, cyclopentyl, cyclobutyl, cyclopropyl, cyclohexenyl, cyclopentenyl, indanyl, tetrahydronaphthyl and the like. The term “carbocyclic” or “carbocylyl”, whether saturated or unsaturated, also refers to rings that are optionally substituted unless indicated. The term “carbocyclic” or “carbocylyl” also encompasses hybrids of aliphatic and carbocyclic groups, such as (cycloalkyl)alkyl, (cycloalkenyl)alkyl and (cycloalkyl)alkenyl.

The term “chemically feasible or stable” refers to a compound structure that is sufficiently stable to allow manufacture and administration to a patient by methods known in the art. Typically, such compounds are stable at a temperature of 40° C. or less, in the absence of moisture or other chemically reactive conditions, for at least one week.

The term “chemical entity” refers to chemical compounds, complexes of at least two chemical compounds, and fragments of such compounds or complexes. The chemical entity can be, for example, a ligand, a substrate, nucleotide triphosphate, a nucleotide, an agonist, antagonist, inhibitor, antibody, peptide, protein or drug. In one embodiment, the chemical entity is selected from the group consisting of an ATP and an inhibitor for the active site. In one embodiment, the inhibitor is 4,5-Diphenyl-1H-pyrazolo[3,4-c]pyridazin-3-ylamine, (5-Methyl-2H-pyrazol-3-yl)-(2-pyridin-4-yl-quinazolin-4-yl)-amine, 4-(5-Methyl-2-phenylamino-pyrimidin-4-yl)-1H-pyrrole-2-carboxylic acid (2-hydroxy-1-phenyl-ethyl)-amide, (1H-Indazol-3-yl)-[2-(2-trifluoromethyl-1-phenyl)-quinazolin-4-yl]-amine and an ATP analogue such as MgAMP-PNP (adenylyl imidodiphosphate) or ADP. In one embodiment, the chemical entity is selected from the group consisting of a peptide substrate or inhibitor for the substrate binding groove.

The term “crystallization solution” refers to a solution that promotes crystallization of macromolecules. The crystallization solution may contain a precipitant, a buffer, salt, stabilizer, a polyionic agent, a detergent, a lanthamide ion or reducing agent. One of ordinary skilled in the art may adjust the components of the crystallization solution to find a condition suitable for the macromolecule of interest.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

The term “generating a three-dimensional structure” refers to plotting the structure coordinates in three-dimensional space. This can be achieved through commercially available software. The three-dimensional structure may be used to perform computer modeling, fitting operations, or displayed as a three-dimensional graphical representation.

The term “kinase inhibitor-binding pocket” or “kinase ATP-binding pocket” refers to a binding pocket of a molecule or molecular complex defined by the structure coordinates of a certain set of amino acid residues present in the kinase structure, as described below. This binding pocket is in an area in the kinase protein where the ATP or inhibitor for the active site binds.

The term “kinase-mediated condition” or “state” refers to any disease or other deleterious condition or state in which kinase is known to play a role. Such diseases or conditions include, without limitation, diabetes, cancer, Alzheimer's disease, Huntington's disease, Parkinson's Disease, AIDS-associated dementia, amyotrophic lateral sclerosis (AML), multiple sclerosis (MS), schizophrenia, cardiomycete hypertrophy, reperfusion/ischemia, and baldness.

The term “halogen” or “halo” means F, Cl, Br, or I.

The term “heteroatom” means N, O, or S and shall include any oxidized form of nitrogen and sulfur, such as N(O), S(O), S(O)₂ and the quaternized form of any basic nitrogen.

The term “heterocyclic” or “heterocyclyl” refers to non-aromatic saturated or unsaturated monocyclic or polycyclic ring systems containing one or more heteroatoms and with a ring size of three to fourteen. One having ordinary skill in the art will recognize that the maximum number of heteroatoms in a stable, chemically feasible heterocyclic ring is determined by the size of the ring, degree of unsaturation, and valence. In general, a heterocyclic ring may have one to four heteroatoms so long as the heterocyclic ring is chemically feasible and stable and may be fused to another ring, such as a carbocyclic, aryl or heteroaryl ring, or to another heterocyclic ring. A heterocyclic ring system may be monocyclic, bicyclic or tricyclic. Also included within the scope of within the scope of the term “heterocyclic” or “heterocyclyl”, as used herein, is a group in which one or more carbocyclic rings are fused to a heteroaryl.

Examples of heterocyclic rings include, but are not limited to, 3-1H-benzimidazol-2-one, 3-1H-alkyl-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxane, benzotriazol-1-yl, benzopyrrolidine, benzopiperidine, benzoxolane, benzothiolane, benzothiane, aziranyl, oxiranyl, azetidinyl, pyrrolinyl, dioxolanyl, imidazolinyl, imidazolidinyl, pyrazolinyl, pyrazolidinyl, pyranyl, dioxanyl, dithianyl, trithianyl, quinuclidinyl, oxepanyl, and thiepanyl. The term “heterocyclic” ring, whether saturated or unsaturated, also refers to rings that are optionally substituted, unless otherwise indicated.

The term “heteroaryl”, alone or in combination with any other term, refers to monocyclic or polycyclic aromatic ring systems having five to fourteen members and one or more heteroatoms. One having ordinary skill in the art will recognize that the maximum number of heteroatoms in a stable, chemically feasible heteroaryl ring is determined by the size of the ring and valence. The term “heteroaralkyl” refers to an alkyl group substituted by a heteroaryl. Also explicitly included within the scope of the term “heteroaralkyl” are alkenyl or alkynyl groups substituted by a heteroaryl. In general, a heteroaryl ring may have one to four heteroatoms. Heteroaryl groups include, without limitation, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 2-pyrrolyl, 3-pyrrolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, and 3-thienyl. The term “heteroaryl ring”, “heteroaryl group” or “heteroaralkyl” also refers to rings that are optionally substituted.

Examples of fused polycyclic heteroaryl and aryl ring systems in which a carbocyclic aromatic ring or heteroaryl ring is fused to one or more other rings include, tetrahydronaphthyl, benzimidazolyl, benzothienyl, benzofuranyl, indolyl, quinolinyl, benzothiazolyl, benzoxazolyl, benzimidazolyl, isoquinolinyl, isoindolyl, acridinyl, benzoisoxazolyl, and the like.

An aryl, aralkyl, heteroaryl, or heteroaralkyl group may contain one or more independently selected substituents. Examples of suitable substituents on the unsaturated carbon atom of an aryl or heteroaryl group include halogen, CF 3, —R′, —OR′, —OH, —SH, —SR′, protected OH (such as acyloxy), —NO₂, —CN, —NH₂, —NHR′, —N(R′)₂, —NHCOR′, —NHCONH₂, —NHCONHR′, —NHCON(R′)₂, —NRCOR′, —NHCO₂H, —NHCO₂R′, —CO₂R′, —CO₂H, —COR′, —CONH₂, —CONHR′, —CON(R′)₂, —S(O)₂H, —S(O)₂R′, —SO₂ NH₂, —S(O)H, —S(O)R′, —SO₂NHR′, —SO₂N(R′)₂, —NHS(O)₂H, or —NHS(O)₂R′, where R′ is selected from H, aliphatic, carbocyclyl, heterocyclyl, aryl, aralkyl, heteroaryl, or heteroaralkyl and each R′ is optionally substituted with halogen, nitro, cyano, amino, —NH-(unsubstituted aliphatic), —N-(unsubstituted aliphatic)₂, carboxy, carbamoyl, hydroxy, —O— (unsubstituted aliphatic), —SH, —S-(unsubstituted aliphatic), CF₃, —SO₂ NH₂, unsubstituted aliphatic, unsubstituted carbocyclyl, unsubstituted heterocyclyl, unsubstituted aryl, unsubstituted aralkyl, unsubstituted heteroaryl, or unsubstituted heteroaralkyl.

An aliphatic group or a non-aromatic heterocyclic ring may contain one or more substituents. Examples of suitable substituents on the saturated carbon of an aliphatic group or of a non-aromatic heterocyclic ring include those listed above for the unsaturated carbon as well as the following: ═O, ═S, ═NNHR′, ═NN(R′)₂, ═N—OR′, ═NNHCOR′, ═NNHCO₂R′, ═NNHSO₂R′, ═N—CN, or ═NR′, wherein R′ is as defined above. Guided by this specification, the selection of suitable substituents is within the knowledge of one skilled in the art.

A substitutable nitrogen on a heteroaryl or a non-aromatic heterocyclic ring is optionally substituted. Suitable substituents on the nitrogen include R″, COR″, S(O)₂R″, and CO₂R″, where R″ is H, an aliphatic group or a substituted aliphatic group.

An “inhibitor” is a molecule (e.g., a small molecule, e.g., less than about 5 kDa in size) that, when it binds to a target (e.g., a kinase), can decrease physiological activity of the target (e.g., render a kinase less functional) or block its activity (e.g., render a kinase non-functional). An inhibitor can be a small molecule of less than 1000 daltons, a small molecule less than 750, 600 or 500 daltons, a polypeptide of naturally occurring or not naturally occurring amino acids, a peptide of naturally occurring or not naturally occurring amino acids, a peptoid, a peptidomimetic, a synthetic compound, a synthetic organic compound, or the like. For example, a target (e.g., an enzyme, e.g., a kinase) which is rendered “less functional” by an inhibitor refers to a target (e.g., an enzyme, e.g., a kinase) having detectable activity which is less than its activity under physiological conditions. A target (e.g., an enzyme, e.g., a kinase) is rendered “non-functional” by an inhibitor if its activity is not detectable by a biological assay (e.g., an enzyme inhibition assay, e.g., a kinase inhibition assay).

The term “motif” refers to a group of amino acids in the protein that defines a structural compartment or carries out a function in the protein, for example, catalysis, structural stabilization or phosphorylation. The motif may be conserved in sequence, structure and function when. The motif can be contiguous in primary sequence or three-dimensional space. Examples of a motif include but are not limited to SXXXS motif, phosphorylation lip or activation loop, the glycine-rich phosphate anchor loop, the catalytic loop, the DFG loop and the APE motif (See, Xie et al., Structure, 6, pp. 983-991 (1998)).

The term “part of a binding pocket” refers to less than all of the amino acid residues that define the binding pocket. The structure coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

The term “pharmaceutically acceptable carrier, adjuvant, or vehicle” refers to a non-toxic carrier, adjuvant, or vehicle that may be administered to a patient, together with a compound of this invention, and which does not destroy the pharmacological activity thereof.

The term “patient” includes human and veterinary subjects.

The term “pharmaceutically acceptable derivative” or “prodrug” means any pharmaceutically acceptable salt, ester, salt of an ester or other derivative of a compound of this invention which, upon administration to a recipient, is capable of providing, either directly or indirectly, a compound of this invention or an inhibitorily active metabolite or residue thereof. Particularly favored derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

The term “protein kinase-mediated condition” or “state” refers to any disease or other deleterious condition or state in which a protein kinase is known to play a role. Such conditions include, without limitation, autoimmune diseases, inflammatory diseases, metabolic, neurological and neurodegenerative diseases, cardiovascular diseases, allergy and asthma.

A “functional site” of a protein refers to any site in a protein that has a function. Representative examples include active sites (i.e., those sites in catalytic proteins where catalysis occurs), protein-protein interaction sites, sites for chemical modification (e.g., glycosylation and phosphorylation sites), and ligand binding sites. Ligand binding sites include metal ion binding sites, co-factor binding sites, antigen binding sites, substrate channels and tunnels, and substrate binding sites.

Accordingly, the present invention provides a compound of formula (I)

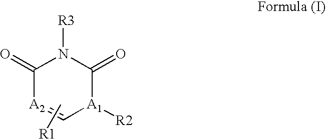

or its stereoisomers, tautomers, solvates, hydrates, pharmaceutically acceptable salts or mixtures thereof, wherein A₁ is Nitrogen; A₂ is Carbon;

R1 is selected from optionally substituted aryl, 5-6 membered heterocyclyl and heteroaryl with 1-3 hetero atoms independently selected from N, O, and S, wherein the substituents are selected from a group consisting of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said 5-6 membered heterocyclyl and heteroaryl exclude nucleosides, sugar moieties, oligomers, peptides, and polymeric ring systems;

R2 and R3 are independently selected from H, alkyl, —(CH₂)ₙ—Y wherein n=0 to 4, and Y is selected from a group consisting of alkyne, alkoxy, ester, carbonyl, carboxyl, aryl, cycloalkyl, heteroaryl and heterocyclyl which are further optionally substituted with halogen, cyano, nitro, alkyl, hydroxyl, alkoxy, carboxyl groups; with the proviso that either R2 or R3 is H and R2 is not same as R3.

Another embodiment of the present invention, is a compound of formula (I) or its stereoisomers, tautomers, solvates, hydrates, pharmaceutically acceptable salts or mixtures thereof, wherein A₁ is Nitrogen; A₂ is Carbon;

R1 is selected from optionally substituted aryl, 5-6 membered heterocyclyl and heteroaryl with 1-3 hetero atoms independently selected from N, O, and S, wherein the substituents are selected from a group comprising of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said 5-6 membered heterocyclyl and heteroaryl exclude nucleosides, sugar moieties, oligomers, peptides, and polymeric ring systems;

R2 and R3 are independently selected from H, alkyl, —(CH₂)ₙ—Y wherein n=0 to 4, and Y is selected from a group consisting of ester, aryl, which are further optionally substituted with 1-5 halogen, cyano, nitro, alkyl, hydroxyl, alkoxy, carbonyl, carboxyl groups; with the proviso that either R2 or R3 is H and R2 is not same as R3.

Yet another embodiment of the present invention, is a compound of formula (I) or its stereoisomers, tautomers, solvates, hydrates, pharmaceutically acceptable salts or mixtures thereof, wherein A₁ is Nitrogen; A₂ is Carbon;

R1 is selected from optionally substituted heteroaryl with 1-3 hetero atoms independently selected from N, O, and S, wherein the substituents are selected from a group comprising of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said heteroaryl excludes nucleosides, sugar moieties, oligomers, peptides, and polymeric ring systems;

R2 and R3 are independently selected from H, alkyl, —(CH₂)ₙ—Y wherein n=0 to 4, and Y is selected from a group consisting of ester, aryl, which are further optionally substituted with 1-5 halogen, cyano, nitro, alkyl, hydroxyl, alkoxy, carboxyl groups; with the proviso that either R2 or R3 is H and R2 is not same as R3.

Further embodiment of the present invention, is a compound of formula (I) or its stereoisomers, tautomers, solvates, hydrates, pharmaceutically acceptable salts or mixtures thereof, wherein A₁ is Nitrogen; A₂ is Carbon;

R1 is a heteroaryl wherein the heteroatom is selected from N, O, and S; with the proviso that the said heteroaryl excludes nucleosides, sugar moieties, oligomers, peptides, and polymeric ring systems;

R2 and R3 are independently selected from H, (CH₂)ₙ—Y wherein n=1 to 4 and Y is selected from a group consisting of ester, aryl, which are further optionally substituted with 1-2 halogen, cyano, nitro, alkyl, hydroxyl, alkoxy, carboxyl groups; with the proviso that either R2 or R3 is H and R2 is not same as R3.

The present invention also provides a preferred compound of formula (I), that is selected from the group consisting of ethyl ester of (2,4-dioxo-5-thiophen-2-yl-3,4-dihydro-2H-pyrimidin-1-yl)acetic acid; 3-(4-nitrobenzyl)-5-furan-2-yl-1H-pyrimidine-2,4-dione; 3-benzyl-5-furan-2-yl-1H-pyrimidine-2,4-dione; 3-(5-furan-2-yl-2,6-dioxo-3,6-dihydro-2H-pyrimidin-1-yl-methyl)benzonitrile; 1-(4-fluorobenzyl)-5-thiophen-2-yl-1H-pyrimidine-2,4-dione; and 3-(4-nitrobenzyl)-5-thiophen-2-yl-1H-pyrimidine-2,4-dione or a pharmaceutically acceptable salt thereof.

In another embodiment of the present invention, wherein preferred compounds of formula (I) are selected from the group consisting of 3-benzyl-5-thiophen-2-yl-1H-pyrimidine-2,4-dione; and 3-(5-thiophen-2-yl-2,6-dioxo-3,6-dihydro-2H-pyrimidin-1yl-methyl)benzonitrile or a pharmaceutically acceptable salt thereof.

In another embodiment of the present invention, wherein preferred compounds of formula (I) are selected from the group consisting of 1-(4-fluorobenzyl)-5-furan-2-yl-1H-pyrimidine-2,4-dione; 3-(3,5-difluorobenzyl)-5-furan-2-yl-1H-pyrimidine-2,4-dione; 4-(5-furan-2-yl-2,6-dioxo-3,6-dihydro-2H-pyrimidin-1yl-methyl)benzonitrile; 3-(4-fluorobenzyl)-5-furan-2-yl-1H-pyrimidine-2,4-dione; 3-(5-thiophen-2-yl-2,6-dioxo-3,6-dihydro-2H-pyrimidin-1-yl-methyl)benzonitrile; and 1-benzyl-5-thiophen-2-yl-1H-pyrimidine-2,4-dione or a pharmaceutically acceptable salt thereof.

In another embodiment of the present invention, wherein preferred compounds of formula (I) are selected from the group consisting of 3-(5-furan-2-yl-2,6-dioxo-3,6-dihydro-2H-pyrimidin-1-yl-methyl)benzonitrile; ethyl ester of (2,6-dioxo-5-furan-2-yl-3,6-dihydro-2H-pyrimidin-1-yl)acetic acid; 4-(5-thiophen-2-yl-2,4-dioxo-3,4-dihydro-2H-pyrimidin-1-yl-methyl)benzonitrile; 4-(5-furan-2-yl-2,6-dioxo-3,6-dihydro-2H-pyrimidin-1-yl-methyl)benzonitrile; ethyl ester of (2,4-dioxo-5-furan-2-yl-3,4-dihydro-2H-pyrimidin-1-yl)acetic acid; and 3-(4-fluorobenzyl)-5-thiophen-2-yl-1H-pyrimidine-2,4-dione or a pharmaceutically acceptable salt thereof.

In an embodiment of the present invention a pharmaceutically acceptable salt of the compound of formula (I) is an acid-addition salt, which is sufficiently basic, such as an inorganic or organic acid or an alkali metal salt, an alkaline earth metal salt.

In another embodiment of the present invention, wherein the compound of formula (I) can exist in the solvated form such as the hydrated form, as well as in the unsolvated form. Accordingly, the present invention encompasses all such forms.

It is of course understood that the compounds of the present invention relate to all optical isomers and stereo-isomers at the various possible atoms. Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Prodrugs and solvates of the compounds of the present invention are also contemplated herein. The term “prodrug”, as employed herein, denotes a compound that is a drug precursor which, upon administration to a subject, undergoes chemical conversion by metabolic or chemical processes to yield a compound of formula (I) or a salt and/or solvate thereof.

The present invention also provides a pharmaceutical composition comprising a compound of formula (I) or a pharmaceutically acceptable salt thereof, in combination with a pharmaceutically acceptable carrier or diluents.

A pharmaceutical composition for use in the prophylaxis or treatment of conditions associated with protein kinase inhibition, comprising, as active ingredient, a pharmaceutically effective amount of the compound of formula (I) in association with pharmaceutically acceptable carriers or diluents.

The process steps for the preparation of compound of formula (I) are described by using the following reaction Scheme A. Further, the process steps of the present invention can also be suitably modified to obtain preferred compounds of compound of formula (I) as hereinafter described.

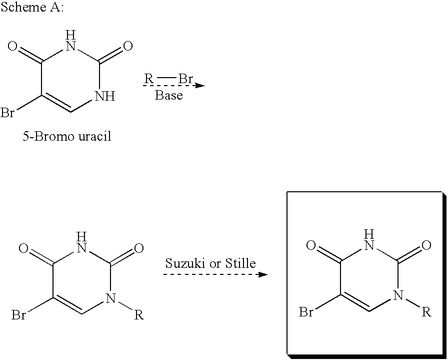

5-halouracil, which upon selective —NH alkylation using basic reaction conditions followed by Suzuki or Stille reaction yields the compound of formula (I).

Alternately, the compound of formula (I) of the present invention is prepared by using reaction scheme B as shown below. The process steps of the present invention can be suitably modified in order to obtain preferred compounds of formula (I) as herein described.

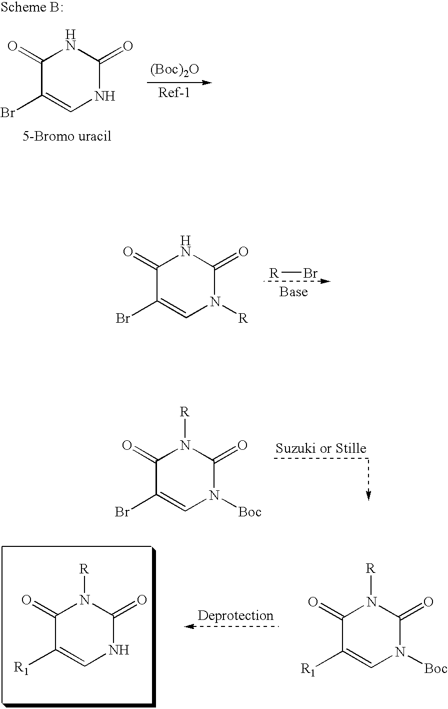

For the desired —NH substitution, comparatively more reactive —NH group of 5-bromouracil is protected with Boc group Alkylation using basic reaction conditions followed by Suzuki or Stille reaction yields and finally deprotection of Boc will yield the desired compounds of the present invention.

The scheme above shows Suzuki Coupling, which is the palladium-catalysed cross coupling between organoboronic acid and halides. This activation of the boron atom enhances the polarisation of the organic ligand, and facilitates transmetallation. If starting materials are substituted with base labile groups (for example esters), powdered KF effects this activation while leaving base labile groups unaffected.

### A. Procedure for Protection of the Active Amino Halo Uracil:—

A two neck round bottom flask was charged with any halo uracil and dry DMF. Sodium hydride was added portion wise in the temperature range of −10° C. to 0° C. and stirred for 15-20 minutes near 0° C. An aryl halide was added drop wise near 0° C. and the reaction mixture was stirred for 1-2 hours at room temperature. It was then poured into ice water and extracted with ethyl acetate. The combined organic layer was washed with brine, dried over sodium sulfate and concentrated under vacuum. The crude product was purified by column chromatography using 2:8 ethyl acetate:dichloromethane.

B. Preparation by Suzuki/Stille Coupling Reaction followed by Deprotection:—

A single neck round bottom flask was charged the protected uracil moiety, active Pd Catalyst [e.g: tributyl-furan-2-yl-stannane, PdCl₂ (PPh₃)₂] and dry DMF. The reaction mixture was refluxed for 10-12 h in the temperature range of 150-180° C. It was then cooled to room temperature, diluted with ethyl acetate and washed with water. The organic layer was stirred with saturated KF solution for 10-12 h, separated and washed with water followed by brine, dried over sodium sulfate and concentrated under vacuum. The crude product was purified by column chromatography using ethyl acetate:hexane in the ratio of 20:80.

In the scheme B as described above, it is understood that protecting groups for sensitive or reactive groups are employed where necessary in accordance with general principles of chemistry. Protecting groups are manipulated according to standard methods of organic synthesis. These groups are removed at a convenient stage of the compound synthesis using methods that are readily apparent to those skilled in the art. The selection of processes as well as the reaction conditions and order of their execution shall be consistent with the preparation of compound of formula (I). Those skilled in the art will recognize if a stereocenter exists in compound of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material needs to be effected by any suitable method known in the art.

The process parameters such as stoichiometry of the reagents, temperature, solvents, etc as indicated above can readily be adapted, to optimize the yield of the desired products.

The present invention also provides a method for the prophylaxis or treatment of a medical condition associated with protein tyrosine kinase, the method comprising administering to a patient in need of such prophylaxis or treatment a pharmaceutically effective amount of the compound of formula (I) or salt thereof.

A method for the prophylaxis or treatment of a medical condition associated with protein kinase can be of dementia, Alzheimer's Disease, Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephalitic parkinsonism, progressive supranuclear palsy, Niemann-Pick's Disease, Pick's Disease, stroke, head trauma and other chronic neurodegenerative diseases, Bipolar Disease, affective disorders, depression, schizophrenia, cognitive disorders, Type I and Type II diabetes, diabetic neuropathy and hair loss.

The method of treating a patient of the present invention, wherein the compounds can be administered alone or in combination with pharmaceutically acceptable agents. When using the compounds, the specific pharmaceutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidentally with the compound used.

The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefore.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

To analyze the activity of the compounds, the said compounds were subjected to biochemical assays. The biochemical assays as performed by the undermentioned methods are used to evaluate the potency of the compounds. This potency data is the indication of the bioactivity of the organic compounds which are described as inhibitors of GSK3beta. The assay used in this method is a standardized protocol and the results thus obtained are tabulated in Table 1.

### GSK3-Beta and GSK3-Alpha Assay Protocol

GSK3 assays were performed using Kinase-Glo® Plus Luminescent Kinase Assay Kit (Promega V3772/3/4 P), in assay buffer (50 mM HEPES, 30 mM Magnesium acetate, 1 mM EDTA and 1 mM EGTA, pH 7.5) in white 96-well-Optiplates (Perkin Elmer). In a typical assay reaction mix (40 μl final volume) contained 10 μl of test compound (dissolved in DMSO), 10 μl of GSM peptide substrate (Upstate Cat#12-533) 20M-GSK3-beta; 25 μM-GSK3-alpha were added followed by 10 μl of enzyme 40 ng/well-GSK3-beta (Upstate Cat#14-306); 30 ng/well-GSK3-alpha (Upstate Cat#14-492) and 1 μM ATP. After incubation at room temperature for 30 minutes, 40 μl of Kinase-Glo reagent was added to stop the reaction, followed by another 5 minutes incubation at room temperature. Luminescence was measured using Wallac 1450 MicroBeta® TriLux (PerkinElmer) Liquid Scintillation and Luminescence Counter. The activity is expressed as a difference of the consumed ATP and total ATP.

The said compounds which are subjected to the biochemical assay exhibited in vitro activity of various ranges as shown in the Table 1.

TABLE 1
Activity Value of GSK3 Beta

| | | | | % | |
|---|---|---|---|---|---|
| | | | | Inhibition | |
| Compound | | n | | at 10 μM | |
| No. | NCE | β | α | β | α |
| 1 | 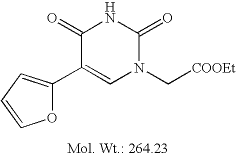 | 4 | 3 | 13 | 9 |
| 2 | 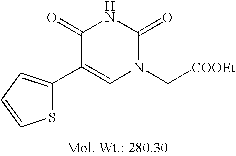 | 4 | 3 | 77 | 68 |
| 3 | 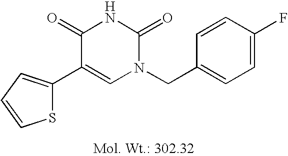 | 4 | 3 | 68 | 11 |
| 4 | 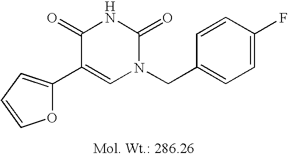 | 4 | 3 | 56 | 72 |
| 5 | 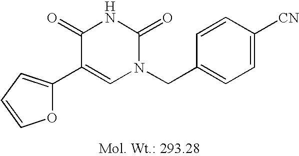 | 4 | 3 | 46 | 17 |
| 6 | 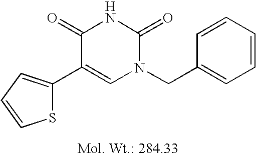 | 4 | 3 | 45 | 10 |
| 7 | 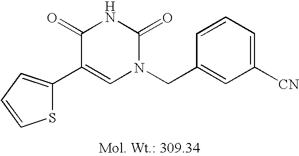 | 4 | 3 | 45 | 16 |
| 8 | 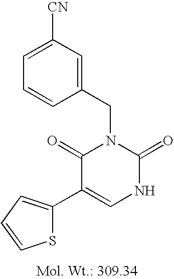 | 4 | 3 | 78 | 54 |
| 9 | 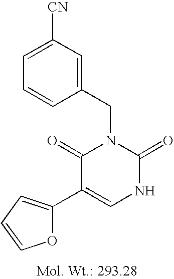 | 4 | 3 | 64 | 22 |
| 10 | 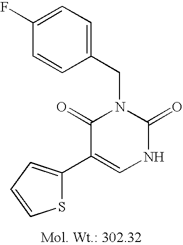 | 4 | 3 | 13 | 6 |
| 11 | 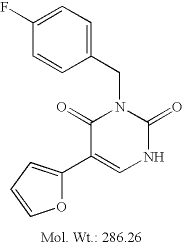 | 4 | 3 | 46 | 16 |
| 12 | 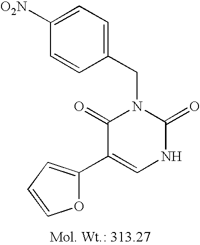 | 4 | 3 | 75 | 55 |
| 13 | 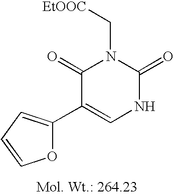 | 4 | 3 | 25 | 13 |
| 14 | 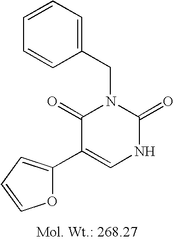 | 4 | 3 | 63 | 42 |
| 15 | 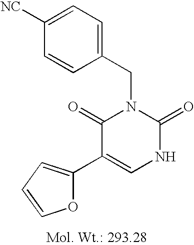 | 4 | 3 | 21 | 14 |
| 16 | 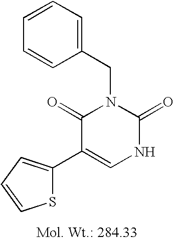 | 4 | 3 | 86 | 64 |
| 17 | 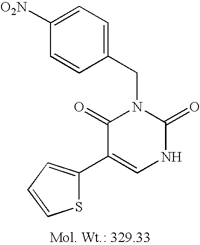 | 4 | 3 | 58 | 8 |
| 18 | 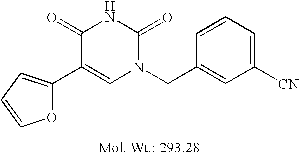 | 4 | 3 | 31 | 10 |
| 19 | 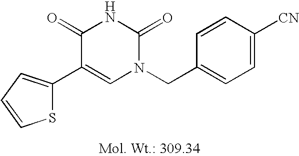 | 4 | 3 | 23 | 15 |
| 20 | 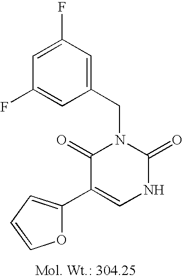 | 4 | 3 | 53 | 11 |

Compound 16 exhibited the highest inhibitory activity of 86% followed by the compound 3 with inhibitory effect of 66%. Interestingly, compound 4 though exhibited an inhibitory effect of 56% for GSK3-beta, but it has shown higher activity of 72% inhibition for GSK3 alpha. This may be due to the difference in the active site residue and the volume of the binding pocket. Compounds 2, 9, 12, 14 and 16 too exhibited higher inhibitory activity of 77%, 75%, 63% respectively.

The above data clearly demonstrates that the compounds developed from the designed scaffolds exhibited high potency for GSK3-beta and same also for GSK3-alpha.

The present invention is now described in connection with certain preferred compounds in the form of following examples. However, these Examples are for illustrative purposes only and shall not be construed as limiting the scope of the present invention. Accordingly, the present invention covers all alternatives, modifications, and equivalents as can be included within the scope of the claims.

### EXAMPLE 1

### (2,4-Dioxo-5-furan-2-yl-3,4-dihydro-2H-pyrimidin-1-yl)-acetic Acid Ethyl Ester

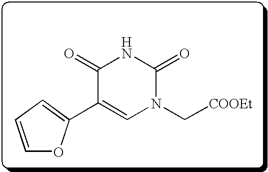

### A. Preparation of (5-bromo-2,4-dioxo-3,4-dihydro-2H-pyrimidin-1-yl)-acetic Acid Ethyl Ester

A 25 ml single neck round bottom flask was charged with 5-bromouracil (2.0 g, 10.4 mmol), potassium carbonate (1.6 g, 11.5 mol) and dry N,N-dimethyl formamide (DMF) (6 ml) at room temperature. Bromo ethyl acetate (2.09 g, 11.9 mmol) was added drop wise and the reaction mixture was stirred for 2 h at room temperature. It was then poured into water and extracted with ethyl acetate (3×30 ml). The combined organic layer was washed with brine, dried over sodium sulfate and concentrated under vacuum. The crude product was purified by column chromatography using 1:9 methanol:dichloromethane. Yield: 1.6 g (55%)

The proton NMR data of the desired product, (5-bromo-2,4-dioxo-3,4-dihydro-2H-pyrimidin-1-yl)-acetic acid ethyl ester, is provided below:

¹H NMR (400 MHz, DMSO-d₆, δppm):

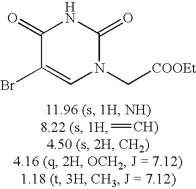

### B. Preparation of (2,4-dioxo-5-furan-2-yl-3,4-dihydro-2H-pyrimidin-1-yl)-acetic Acid Ethyl Ester

A 25 ml single neck round bottom flask was charged with (5-Bromo-2,4-dioxo-3,4-dihydro-2H-pyrimidin-1-yl)-acetic acid ethyl ester (400 mg, 1.44 mmol), tributyl-furan-2-yl-stannane (618 mg, 1.73 mmol), PdCl₂(PPh₃)₂ (50 mg, 0.07 mmol) and dry DMF (5 ml). The reaction mixture was refluxed for 12 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=290 mg (76%)

The proton NMR data of the desired product, (2,4-dioxo-5-furan-2-yl-3,4-dihydro-2H-pyrimidin-1-yl)-acetic acid ethyl ester, is provided below:

¹H NMR (400 MHz, DMSO-d₆, δppm):

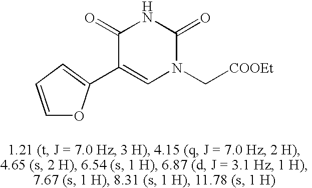

### EXAMPLE 2

### (2,4-Dioxo-5-thiophen-2-yl-3,4-dihydro-2H-pyrimidin-1-yl)-acetic acid Ethyl Ester

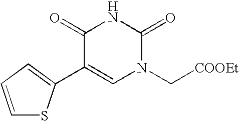

### A. Preparation of (5-bromo-2,4-dioxo-3,4-dihydro-2H-pyrimidin-1-yl)-acetic Acid Ethyl Ester

A 25 ml single neck round bottom flask was charged with 5-bromouracil (2.0 g, 10.4 mmol), potassium carbonate (1.6 g, 11.5 mol) and dry DMF (6 ml) at room temperature. Bromo ethyl acetate (2.09 g, 11.9 mmol) was added drop wise and the reaction mixture was stirred for 2 h at room temperature. It was then poured into water and extracted with ethyl acetate (3×30 ml). The combined organic layer was washed with brine, dried over sodium sulfate and concentrated under vacuum. The crude product was purified by column chromatography using 1:9 methanol:dichloromethane.

Yield=1.6 g (55%)

The proton NMR data of the desired product, (5-bromo-2,4-dioxo-3,4-dihydro-2H-pyrimidin-1-yl)-acetic acid ethyl ester, is provided below:

¹H NMR (400 MHz, DMSO-d₆, δppm):

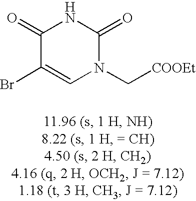

### B. Preparation of (2,4-dioxo-5-thiophen-2-yl-3,4-dihydro-2H-pyrimidin-1-yl)-acetic Acid Ethyl Ester

A 25 ml single neck round bottom flask was charged with (5-Bromo-2,4-dioxo-3,4-dihydro-2H-pyrimidin-1-yl)-acetic acid ethyl ester (400 mg, 1.44 mmol), tributyl-thiophene-2-yl-stannane (646 mg, 1.73 mmol), PdCl₂(PPh₃)₂ (50 mg, 0.07 mmol) and dry DMF (5 ml). The reaction mixture was refluxed for 12 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=100 mg (25%)

The proton NMR data of the desired product, (2,4-dioxo-5-thiophen-2-yl-3,4-dihydro-2H-pyrimidin-1-yl)-acetic acid ethyl ester, is provided below:

¹H NMR (400 MHz, DMSO-d₆, δppm):

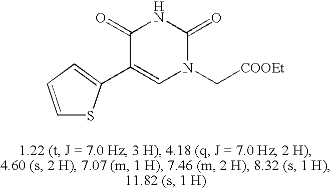

### EXAMPLE 3

### 1-(4-Fluoro-benzyl)-5-thiophen-2-yl-1H-pyrimidine-2,4-dione

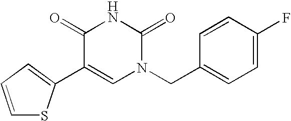

### A. Preparation of 5-iodo-1-(4-fluoro-benzyl)-1H-pyrimidine-2,4-dione

A 25 ml two neck round bottom flask was charged with 5-iodouracil (3.0 g, 12.6 mmol) and dry DMF (10 ml). Sodium hydride (363 mg, 15.1 mmol) was added portion wise at −5° C. and stirred for 15 min at 0° C. 4-Fluorobenzyl bromide (2.7 g, 14.2 mmol) was added drop wise at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane. Yield=1.0 g (23%)

The proton NMR data of the desired product, 5-iodo-1-(4-fluoro-benzyl)-1H-pyrimidine-2,4-dione, is provided below:

¹H NMR (400 MHz, DMSO-d₆, δppm):

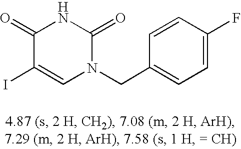

### B. Preparation of 1-(4-fluoro-benzyl)-5-thiophen-2-yl-1H-pyrimidine-2,4-dione

A 25 ml single neck round bottom flask was charged with 5-iodo-1-(4-fluoro-benzyl)-1H-pyrimidine-2,4-dione (500 mg, 1.2 mmol), tributyl-thiophene-2-yl-stannane (551 mg, 1.47 mmol), PdCl₂(PPh₃)₂ (43 mg, 0.06 mmol) and dry DMF (5 ml). The reaction mixture was refluxed for 12 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=130 mg (29%)

The proton NMR data of the desired product, 1-(4-fluoro-benzyl)-5-thiophen-2-yl-1H-pyrimidine-2,4-dione, is provided below:

¹H NMR (400 MHz, DMSO-d₆, δppm):

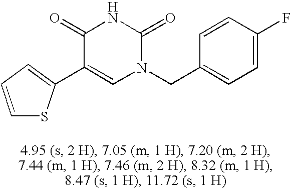

### EXAMPLE 4

### 1-(4-Fluoro-benzyl)-5-furan-2-yl-1H-pyrimidine-2,4-dione

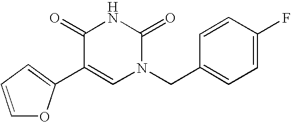

### A. Preparation of 5-iodo-1-(4-fluoro-benzyl)-1H-pyrimidine-2,4-dione

A 25 ml two neck round bottom flask was charged with 5-iodouracil (3.0 g, 12.6 mmol) and dry DMF (10 ml). Sodium hydride (363 mg, 15.1 mmol) was added portion wise at −5° C. and stirred for 15 min at 0° C. 4-Fluorobenzyl bromide (2.7 g, 14.2 mmol) was added drop wise at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane.

Yield=1.0 g (23%)

The proton NMR data of the desired product, 5-iodo-1-(4-fluoro-benzyl)-1H-pyrimidine-2,4-dione, is provided below:

¹H NMR (400 MHz, DMSO-d₆, δppm):

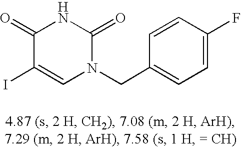

### B. Preparation of 1-(4-fluoro-benzyl)-5-furan-2-yl-1H-pyrimidine-2,4-dione

A 25 ml single neck round bottom flask was charged with 5-iodo-1-(4-fluoro-benzyl)-1H-pyrimidine-2,4-dione (400 mg, 0.98 mmol), tributyl-furan-2-yl-stannane (422 mg, 1.18 mmol), PdCl₂(PPh₃)₂ (34 mg, 0.05 mmol) and dry DMF (5 ml). The reaction mixture was refluxed for 12 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=65 mg (20%)

The proton NMR data of the desired product, 1-(4-fluoro-benzyl)-5-furan-2-yl-1H-pyrimidine-2,4-dione, is provided below:

¹H NMR (400 MHz, DMSO-d₆, δppm):

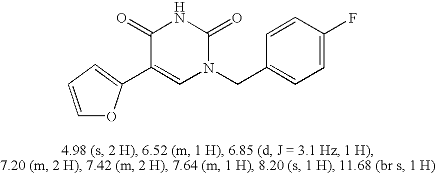

### EXAMPLE 5

### 4-(2,4-Dioxo-5-furan-2-yl-3,4-dihydro-2H-pyrimidin-1-ylmethyl)-benzonitrile

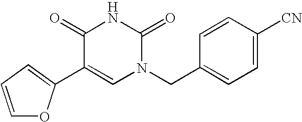

### A. Preparation of 5-iodo-1-(4-cyanobenzyl)-1H-pyrimidine-2,4-dione

A 25 ml two neck round bottom flask was charged with 5-iodouracil (1.5 g, 6.3 mmol) and dry DMF (10 ml). Sodium hydride (181 mg, 7.56 mmol) was added portion wise at −5° C. and stirred for 15 min at 0° C. 4-Cyanobenzyl bromide (1.5 g, 7.56 mmol) was added at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane. Yield=230 mg (10.3%)

The proton NMR data of the desired product, 5-iodo-1-(4-cyanobenzyl)-1H-pyrimidine-2,4-dione, is provided below:

¹H NMR (400 MHz, DMSO-d₆, δppm):

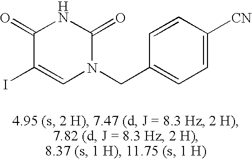

### B. Preparation of 4-(2,4-dioxo-5-furan-2-yl-3,4-dihydro-2H-pyrimidin-1-ylmethyl)-benzonitrile

A 25 ml single neck round bottom flask was charged with 5-iodo-1-(4-cyano-benzyl)-1H-pyrimidine-2,4-dione (230 mg, 0.89 mmol), tributyl-furan-2-yl-stannane (381 mg, 1.0 mmol), PdCl₂(PPh₃)₂ (31 mg, 0.04 mmol) and dry DMF (5 ml). The reaction mixture was refluxed for 12 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=60 mg (17%)

The proton NMR data of the desired product, 4-(2,4-dioxo-5-furan-2-yl-3,4-dihydro-2H-pyrimidin-1-ylmethyl)-benzonitrile, is provided below:

¹H NMR (400 MHz, DMSO-d₆, δppm):

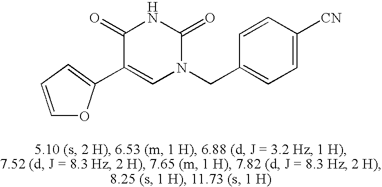

### EXAMPLE 6

### 1-Benzyl-5-thiophen-2-yl-1H-pyrimidine-2,4-dione

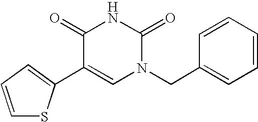

### A. Preparation of 1-benzyl-5-bromo-1H-pyrimidine-2,4-dione

A 25 ml two neck round bottom flask was charged with 5-bromouracil (3.0 g, 15.7 mmol) and dry DMF (10 ml). Sodium hydride (432 mg, 18.0 mmol) was added portion wise at −5° C. and stirred for 15 min at 0° C. Benzyl bromide (3.0 g, 18.0 mmol) was added drop wise at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane. Yield=1.2 g (27%)

The proton NMR data of the desired product, 1-benzyl-5-bromo-1H-pyrimidine-2,4-dione, is provided below:

¹H NMR (400 MHz, DMSO-d₆, δppm):

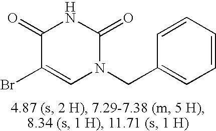

### B. Preparation of 1-benzyl-5-thiophen-2-yl-1H-pyrimidine-2,4-dione

A 25 ml single neck round bottom flask was charged with 1-benzyl-5-bromo-1H-pyrimidine-2,4-dione (250 mg, 0.76 mmol), tributyl-thiophene-2-yl-stannane (341 mg, 0.91 mmol), PdCl₂(PPh₃)₂ (27 mg, 0.038 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=93 mg (43%)

The proton NMR data of the desired product, 1-benzyl-5-thiophen-2-yl-1H-pyrimidine-2,4-dione, is provided below:

¹H NMR (400 MHz, DMSO-d₆, δppm):

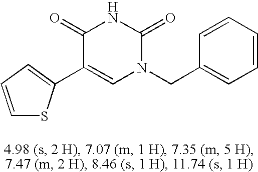

### EXAMPLE 7

### 3-(2,4-dioxo-5-thiophen-2-yl-3,4-dihydro-2H-pyrimidin-1-ylmethyl)-benzonitrile

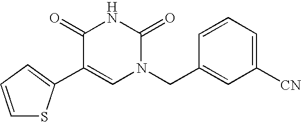

### A. Preparation of 3-(5-iodo-2,4-dioxo-3,4-dihydro-2H-pyrimidin-1-ylmethyl)-benzonitrile

A 25 ml two neck round bottom flask was charged with 5-iodouracil (3.0 g, 12.6 mmol) and dry DMF (10 ml). Sodium hydride (362 mg, 15.1 mmol) was added portion wise at −5° C. and stirred for 15 min at 0° C. 3-Cyanobenzyl bromide (2.9 g, 15.1 mmol) was added drop wise at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane. Yield=1.2 g (27%)

The proton NMR data of the desired product, 3-(5-iodo-2,4-dioxo-3,4-dihydro-2H-pyrimidin-1-ylmethyl)-benzonitrile, is provided below:

¹H NMR (400 MHz, DMSO-d₆, δppm):

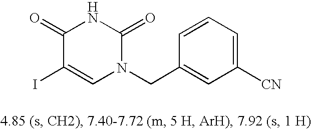

### B. Preparation of 3-(2,4-dioxo-5-thiophen-2-yl-3,4-dihydro-2H-pyrimidin-1-ylmethyl)-benzonitrile

A 25 ml single neck round bottom flask was charged with 3-(5-iodo-2,4-dioxo-3,4-dihydro-2H-pyrimidin-1-ylmethyl)-benzonitrile (500 mg, 1.46 mmol), tributyl-thiophene-2-yl-stannane (556 mg, 1.49 mmol), PdCl₂(PPh₃)₂ (49 mg, 0.06 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=60 mg (14%)

The proton NMR data of the desired product, 3-(2,4-dioxo-5-thiophen-2-yl-3,4-dihydro-2H-pyrimidin-1-ylmethyl)-benzonitrile, is provided below:

¹H NMR (400 MHz, DMSO-d₆, δppm):

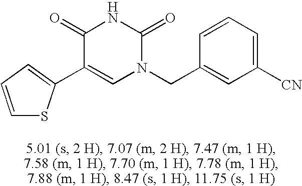

### EXAMPLE 8

### 3-(2,6-Dioxo-5-thiophen-2-yl-3,6-dihydro-2H-pyrimidin-1-ylmethyl)-benzonitrile

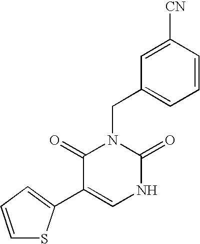

### A. Preparation of 5-iodo-2,4-dioxo-3,4-dihydro-2H-pyrimidine-1-carboxylic Acid Tert-Butyl Ester

A 500 ml single neck round bottom flask was charged with 5-iodouracil (5.0 g, 21 mmol) and dry acetonitrile (200 ml). Dimethyl amino pyridine (26 mg, 0.21 mmol) was added in one portion followed by drop wise addition of di-t-butyl dicarbonate (5.5 g, 25.2 mmol) at room temperature and stirred for 3 h at room temperature. The reaction mixture was filtered and the organic layer was concentrated under vacuum.

Yield=7.1 (100%)

The proton NMR data of the desired product, 5-iodo-2,4-dioxo-3,4-dihydro-2H-pyrimidine-1-carboxylic acid tert-butyl ester, is provided below:

¹H NMR (400 MHz, DMSO-d₆, δppm):

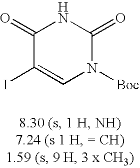

### B. Preparation of 3-(5-iodo-2,6-dioxo-3,6-dihydro-2H-pyrimidin-1-ylmethyl)-benzonitrile

A 25 ml two neck round bottom flask was charged with 5-iodo-2,4-dioxo-3,4-dihydro-2H-pyrimidine-1-carboxylic acid tert-butyl ester (2.0 g, 5.93 mmol) and dry DMF (10 ml). Sodium hydride (170 mg, 7.12 mmol) was added portion wise at −5° C. and stirred for 15 min at 0° C. 3-Cyanobenzyl bromide (1.39 g, 7.1 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=837 mg (40.0%)

The proton NMR data of the desired product, 3-(5-iodo-2,6-dioxo-3,6-dihydro-2H-pyrimidin-1-ylmethyl)-benzonitrile, is provided below:

¹H NMR (400 MHz, DMSO-d₆; δppm):

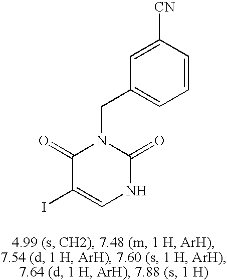

### C. Preparation of 3-(2,6-dioxo-5-thiophen-2-yl-3,6-dihydro-2H-pyrimidin-1-ylmethyl)-benzonitrile

A 25 ml single neck round bottom flask was charged with 3-(5-Iodo-2,6-dioxo-3,6-dihydro-2H-pyrimidin-1-ylmethyl)-benzonitrile (300 mg, 0.85 mmol), tributyl-thiophene-2-yl-stannane (348 mg, 0.93 mmol), PdCl₂(PPh₃)₂ (29 mg, 0.04 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=50 mg (19%)

The proton NMR data of the desired product, 3-(2,6-dioxo-5-thiophen-2-yl-3,6-dihydro-2H-pyrimidin-1-ylmethyl)-benzonitrile, is provided below:

¹H NMR (400 MHz, DMSO-d₆, δppm):

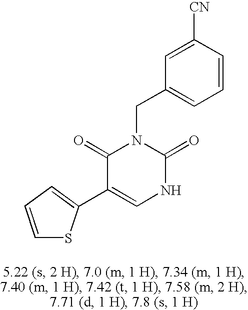

### EXAMPLE 9

### 3-(5-furan-2-yl-2,6-dioxo-3,6-dihydro-2H-pyrimidin-1-ylmethyl)-benzonitrile

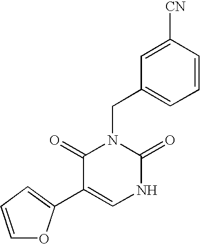

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-(5-iodo-2,6-dioxo-3,6-dihydro-2H-pyrimidin-1-ylmethyl)-benzonitrile

A 25 ml two neck round bottom flask was charged with 5-iodo-2,4-dioxo-3,4-dihydro-2H-pyrimidine-1-carboxylic acid tert-butyl ester (2.0 g, 5.93 mmol) and dry DMF (10 ml). Sodium hydride (170 mg, 7.12 mmol) was added portion wise at −5° C. and stirred for 15 min at 0° C. 3-Cyanobenzyl bromide (1.39 g, 7.1 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=837 mg (40.0%)

The proton NMR data of the desired product, 3-(5-iodo-2,6-dioxo-3,6-dihydro-2H-pyrimidin-1-ylmethyl)-benzonitrile, is provided below:

¹H NMR (400 MHz, DMSO-d₆, δppm):

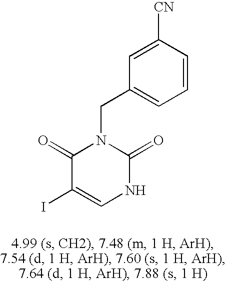

### C. Preparation of 3-(5-furan-2-yl-2,6-dioxo-3,6-dihydro-2H-pyrimidin-1-ylmethyl)-benzonitrile

A 25 ml single neck round bottom flask was charged with 3-(5-iodo-2,6-dioxo-3,6-dihydro-2H-pyrimidin-1-ylmethyl)-benzonitrile (300 mg, 0.85 mmol), tributyl-furan-2-yl-stannane (330 mg, 0.92 mmol), PdCl₂(PPh₃)₂ (29 mg, 0.042 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=56 mg (22.4%)

The proton NMR data of the desired product, 3-(5-furan-2-yl-2,6-dioxo-3,6-dihydro-2H-pyrimidin-1-ylmethyl)-benzonitrile, is provided below:

¹H NMR (400 MHz, DMSO-d₆, δppm):

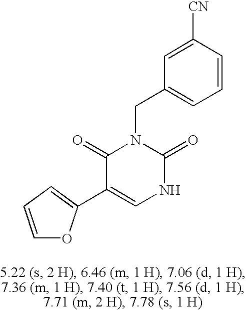

### EXAMPLE 10

### 3-(4-fluoro-benzyl)-5-thiophen-2-yl-1H-pyrimidine-2,4-dione

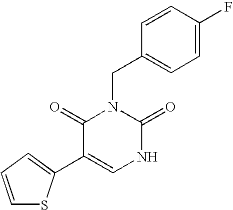

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-(4-fluoro-benzyl)-5-iodo-1H-pyrimidine-2,4-dione

A 25 ml two neck round bottom flask was charged with 5-iodo-2,4-dioxo-3,4-dihydro-2H-pyrimidine-1-carboxylic acid tert-butyl ester (1.0 g, 2.9 mmol) and dry DMF (10 ml). Sodium hydride (85 mg, 3.5 mmol) was added portion wise at −5° C. and stirred for 15 min at 0° C. 4-Fluorobenzyl bromide (70 mg, 3.5 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (410 mg, 2.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=310 mg (35%)

The proton NMR data of the desired product, 3-(4-fluoro-benzyl)-5-iodo-1H-pyrimidine-2,4-dione, is provided below:

¹H NMR (400 MHz, DMSO-d₆, δppm):

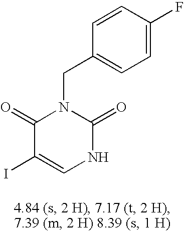

### C. Preparation of 3-(4-fluoro-benzyl)-5-thiophen-2-yl-1H-pyrimidine-2,4-dione

A 25 ml single neck round bottom flask was charged with 3-(4-fluoro-benzyl)-5-iodo-1H-pyrimidine-2,4-dione (200 mg, 0.66 mmol), tributyl-thiophene-2-yl-stannane (270 mg, 0.72 mmol), PdCl₂(PPh₃)₂ (23 mg, 0.033 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=78 mg (39%)

The proton NMR data of the desired product, 3-(4-fluoro-benzyl)-5-thiophen-2-yl-1H-pyrimidine-2,4-dione, is provided below:

¹H NMR (400 MHz, DMSO-d₆, δppm):

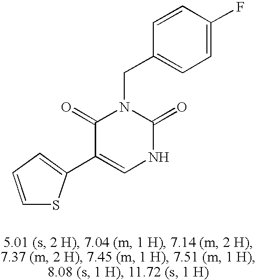

### EXAMPLE 11

### 3-(4-fluoro-benzyl)-5-furan-2-yl-1H-pyrimidine-2,4-dione

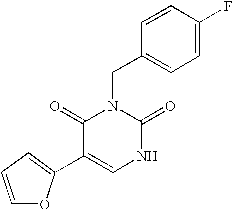

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-(4-fluoro-benzyl)-5-iodo-1H-pyrimidin-2,4-dione

A 25 ml two neck round bottom flask was charged with 5-iodo-2,4-dioxo-3,4-dihydro-2H-pyrimidine-1-carboxylic acid tert-butyl ester (1.0 g, 2.9 mmol) and dry DMF (10 ml). Sodium hydride (85 mg, 3.5 mmol) was added portion wise at −5° C. and stirred for 15 min at 0° C. 4-Fluorobenzyl bromide (70 mg, 3.5 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (410 mg, 2.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=310 mg (35%)

The proton NMR data of the desired product, 3-(4-fluoro-benzyl)-5-iodo-1H-pyrimidin-2,4-dione, is provided below:

¹H NMR (400 MHz, DMSO-d₆, δppm):

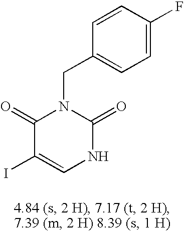

### C. Preparation of 3-(4-fluoro-benzyl)-5-furan-2-yl-1H-pyrimidine-2,4-dione

A 25 ml single neck round bottom flask was charged with 3-(4-fluoro-benzyl)-5-iodo-1H-pyrimidin-2,4-dione (200 mg, 0.57 mmol), tributyl-furan-2-yl-stannane (240 mg, 0.67 mmol), PdCl₂(PPh₃)₂ (21 mg, 0.03 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=32 mg (14%).

The proton NMR data of the desired product, 3-(4-fluoro-benzyl)-5-furan-2-yl-1H-pyrimidine-2,4-dione, is provided below:

¹H NMR (400 MHz, DMSO-d₆, δppm):

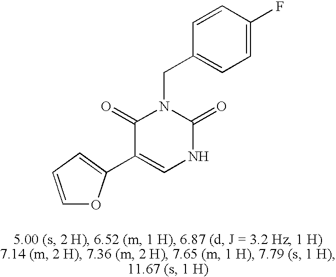

### EXAMPLE 12

### 3-(4-nitro-benzyl)-5-furan-2-yl-1H-pyrimidine-2,4-dione

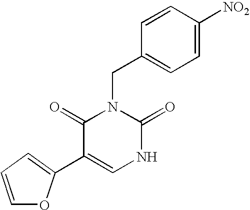

### A. Preparation of 5-iodo-2,4-dioxo-3,4-dihydro-2H-pyrimidin-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 5-iodo-3-(4-nitro-benzyl)-1H-pyrimidine-2,4-dione

A 25 ml two neck round bottom flask was charged with 5-iodo-2,4-dioxo-3,4-dihydro-2H-pyrimidin-1-carboxylic acid tert-butyl ester (3.0 g, 10.3 mmol) and dry DMF (10 ml). Sodium hydride (295 mg, 12.3 mmol) was added portion wise at −5° C. and stirred for 15 min at 0° C. 4-Nitrobenzyl bromide (2.65 g, 12.3 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (1.42 g, 10.3 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80.

Yield=640 mg (50%)

The proton NMR data of the desired product, 5-iodo-3-(4-nitro-benzyl)-1H-pyrimidine-2,4-dione, is provided below:

¹H NMR (400 MHz, DMSO-d₆, δppm):

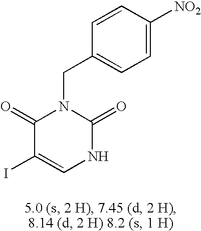

### C. Preparation of 3-(4-nitro-benzyl)-5-furan-2-yl-1H-pyrimidine-2,4-dione

A 25 ml single neck round bottom flask was charged with 5-iodo-3-(4-nitro-benzyl)-1H-pyrimidine-2,4-dione (400 mg, 1.07 mmol), tributyl-furan-2-yl-stannane (420 mg, 1.17 mmol), PdCl₂(PPh₃)₂ (37 mg, 0.05 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=30 mg (9%)

The proton NMR data of the desired product, 3-(4-nitro-benzyl)-5-furan-2-yl-1H-pyrimidine-2,4-dione, is provided below:

¹H NMR (400 MHz, DMSO-d₆, δppm):

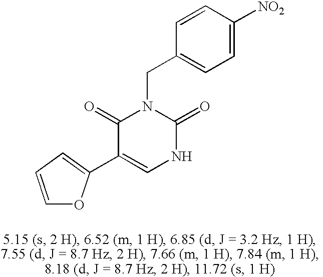

### EXAMPLE 13

### (2,6-dioxo-5-furan-2-yl-3,6-dihydro-2H-pyrimidin-1-yl)-acetic Acid Ethyl Ester

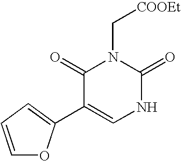

### A. Preparation of 5-iodo-2,4-dioxo-3,4-dihydro-2H-pyrimidin-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of (5-iodo-2,6-dioxo-3,6-dihydro-2H-pyrimidin-1-yl)-acetic Acid Ethyl Ester

A 25 ml two neck round bottom flask was charged with 5-iodo-2,4-dioxo-3,4-dihydro-2H-pyrimidin-1-carboxylic acid tert-butyl ester (2.0 g, 6.8 mmol) and dry DMF (10 ml). Sodium hydride (200 mg, 8.3 mmol) was added portion wise at −5° C. and stirred for 15 min at 0° C. Ethyl bromo acetate (1.37 g, 8.3 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (940 mg, 6.8 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=900 mg (45%)

The proton NMR data of the desired product, (5-iodo-2,6-dioxo-3,6-dihydro-2H-pyrimidin-1-yl)-acetic acid ethyl ester, is provided below:

¹H NMR (400 MHz, DMSO-d₆, δppm):

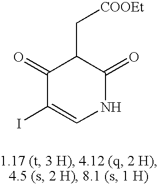

### C. Preparation of (2,6-dioxo-5-furan-2-yl-3,6-dihydro-2H-pyrimidin-1-yl)-acetic Acid Ethyl Ester

A 25 ml single neck round bottom flask was charged with (5-iodo-2,6-dioxo-3,6-dihydro-2H-pyrimidin-1-yl)-acetic acid ethyl ester (375 mg, 1.35 mmol), tributyl-furan-2-yl-stannane (531 mg, 1.48 mmol), PdCl₂(PPh₃)₂ (47 mg, 0.06 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=100 mg (28%)

The proton NMR data of the desired product, (2,6-dioxo-5-furan-2-yl-3,6-dihydro-2H-pyrimidin-1-yl)-acetic acid ethyl ester, is provided below:

¹H NMR (400 MHz, DMSO-d₆, δppm):

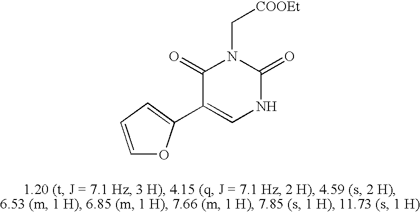

### EXAMPLE 14

### 3-benzyl-5-furan-2-yl-1H-pyrimidine-2,4-dione

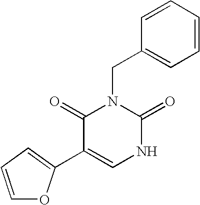

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-benzyl-5-iodo-1H-pyrimidin-2,4-dione

A 25 ml two neck round bottom flask was charged with 5-iodo-2,4-dioxo-3,4-dihydro-2H-pyrimidine-1-carboxylic acid tert-butyl ester (2.0 g, 5.8 mmol) and dry DMF (10 ml). Sodium hydride (169 mg, 7.0 mmol) was added portion wise at −5° C. and stirred for 15 min at 0° C. Benzyl bromide (1.0 g, 5.8 mmol) was added drop wise at 0° C. and the reaction mixture was stirred for 3 h at room temperature. Reaction mixture was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (800 mg, 5.8 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography in the ratio of 20:80. Yield=970 mg (47%)

The proton NMR data of the desired product, 3-benzyl-5-iodo-1H-pyrimidin-2,4-dione, is provided below:

¹H NMR (400 MHz, DMSO-d₆, δppm):

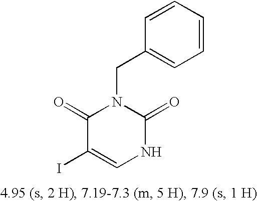

### C. Preparation of 3-benzyl-5-furan-2-yl-1H-pyrimidine-2,4-dione

A 25 ml single neck round bottom flask was charged with 3-benzyl-5-iodo-1H-pyrimidin-2,4-dione (200 mg, 0.6 mmol), tributyl-furan-2-yl-stannane (200 mg, 0.56 mmol), PdCl₂(PPh₃)₂ (21 mg, 0.03 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=75 mg (46%)

The proton NMR data of the desired product, 3-benzyl-5-furan-2-yl-1H-pyrimidine-2,4-dione, is provided below:

¹H NMR (400 MHz, DMSO-d₆, δppm):

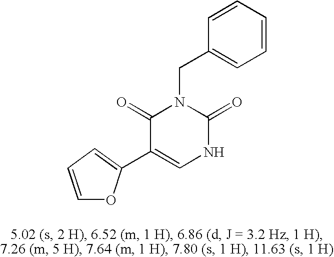

### EXAMPLE 15

### 4-(2,6-dioxo-5-furan-2-yl-3,6-dihydro-2H-pyrimidin-1-ylmethyl)-benzonitrile

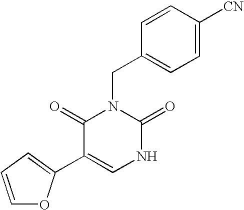

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 4-(5-iodo-2,6-dioxo-3,6-dihydro-2H-pyrimidin-1-ylmethyl)-benzonitrile

A 25 ml two neck round bottom flask was charged with 5-iodo-2,4-dioxo-3,4-dihydro-2H-pyrimidine-1-carboxylic acid tert-butyl ester (2.0 g, 5.8 mmol) and dry DMF (10 ml). Sodium hydride (171 mg, 7.1 mmol) was added portion wise at −5° C. and stirred for 15 min at 0° C. 4-Cyanobenzyl bromide (1.38 g, 7.1 mmol) was added drop wise at 0° C. and the reaction mixture was stirred for 3 h at room temperature. Reaction mixture was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=855 mg (42%)

The proton NMR data of the desired product, 4-(5-iodo-2,6-dioxo-3,6-dihydro-2H-pyrimidin-1-ylmethyl)-benzonitrile, is provided below:

¹H NMR (400 MHz, DMSO-d₆, δppm):

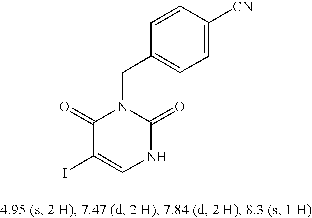

### C. Preparation of 4-(2,6-dioxo-5-furan-2-yl-3,6-dihydro-2H-pyrimidin-1-ylmethyl)-benzonitrile

A 25 ml single neck round bottom flask was charged with 4-(5-Iodo-2,6-dioxo-3,6-dihydro-2H-pyrimidin-1-ylmethyl)-benzonitrile (220 mg, 6.23 mmol), tributyl-furan-2-yl-stannane (266 mg, 7.4 mmol), PdCl₂(PPh₃)₂ (22 mg, 0.03 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=75 mg (41%)

The proton NMR data of the desired product, 4-(2,6-dioxo-5-furan-2-yl-3,6-dihydro-2H-pyrimidin-1-ylmethyl)-benzonitrile, is provided below:

¹H NMR (400 MHz, DMSO-d₆, δppm):

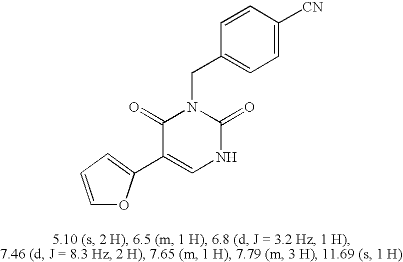

### EXAMPLE 16

### 3-(4-fluoro-benzyl)-5-furan-2-yl-1H-pyrimidine-2,4-dione

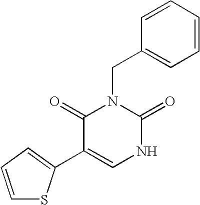

### A. Preparation of 5-iodo-2,4-dioxo-3,4-dihydro-2H-pyrimidin-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-benzyl-5-iodo-1H-pyrimidin-2,4-dione

A 25 ml two neck round bottom flask was charged with 5-iodo-2,4-dioxo-3,4-dihydro-2H-pyrimidin-1-carboxylic acid tert-butyl ester (2.0 g, 5.8 mmol) and dry DMF (10 ml). Sodium hydride (169 mg, 7.0 mmol) was added portion wise at −5° C. and stirred for 15 min at 0° C. Benzyl bromide (1.0 g, 5.8 mmol) was added drop wise at 0° C. and the reaction mixture was stirred for 3 h at room temperature. Reaction mixture was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=970 mg (47%)

The proton NMR data of the desired product, 3-benzyl-5-iodo-1H-pyrimidin-2,4-dione, is provided below:

¹H NMR (400 MHz, DMSO-d₆, δppm):

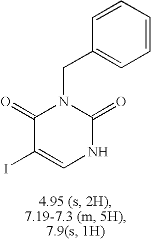

### C. Preparation of 3-(4-fluoro-benzyl)-5-furan-2-yl-1H-pyrimidine-2,4-dione

A 25 ml single neck round bottom flask was charged with 3-Benzyl-5-iodo-1H-pyrimidin-2,4-dione (200 mg, 0.6 mmol), tributyl-thiophene-2-yl-stannane (22 mg, 0.60 mmol), PdCl₂(PPh₃)₂ (21 mg, 0.03 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=60 mg (34%)

The proton NMR data of the desired product, 3-(4-fluoro-benzyl)-5-furan-2-yl-1H-pyrimidine-2,4-dione, is provided below:

¹H NMR (400 MHz, DMSO-d₆, δppm):

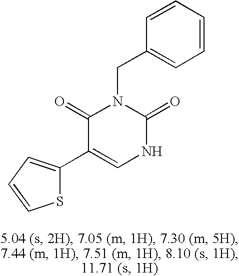

### EXAMPLE 17

### 3-(4-nitro-benzyl)-5-thiophen-2-yl-1H-pyrimidine-2,4-dione

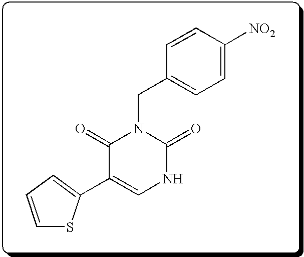

### A. Preparation of 5-iodo-2,4-dioxo-3,4-dihydro-2H-pyrimidine-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 5-iodo-3-(4-nitro-benzyl)-1H-pyrimidine-2,4-dione

A 25 ml two neck round bottom flask was charged with 5-iodo-2,4-dioxo-3,4-dihydro-2H-pyrimidine-1-carboxylic acid tert-butyl ester (2.0 g, 5.9 mmol) and dry DMF (10 ml). Sodium hydride (169 mg, 7.0 mmol) was added portion wise at −5° C. and stirred for 15 min at 0° C. 4-Nitrobenzyl bromide (1.53 g, 7.0 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=640 mg (50%)

The proton NMR data of the desired product, 5-iodo-3-(4-nitro-benzyl)-1H-pyrimidine-2,4-dione, is provided below:

¹H NMR (400 MHz, DMSO-d₆, δppm):

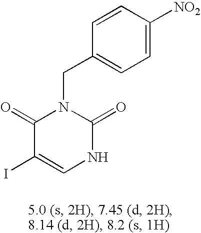

### C. Preparation of 3-(4-nitro-benzyl)-5-thiophen-2-yl-1H-pyrimidine-2,4-dione

A 25 ml single neck round bottom flask was charged with 5-iodo-3-(4-nitro-benzyl)-1H-pyrimidine-2,4-dione (350 mg, 0.93 mmol), tributyl-thiophene-2-yl-stannane (420 mg, 1.1 mmol), PdCl₂(PPh₃)₂ (32 mg, 0.04 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column using ethyl acetate:hexane in the ratio of 20:80. Yield=35 mg (11%)

The proton NMR data of the desired product, 3-(4-nitro-benzyl)-5-thiophen-2-yl-1H-pyrimidine-2,4-dione, is provided below:

¹H NMR (400 MHz, DMSO-d₆, δppm):

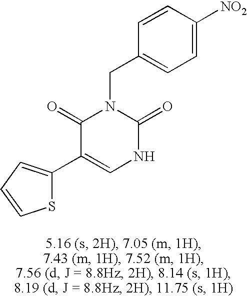

### EXAMPLE 18

### 3-(5-furan-2-yl-2,4-dioxo-3,4-dihydro-2H-pyrimidin-1-ylmethyl)-benzonitrile

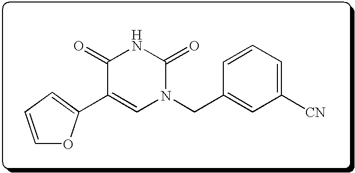

### A. Preparation of 1-benzyl-5-iodo-1H-pyrimidine-2,4-dione

A 25 ml two neck round bottom flask was charged with 5-iodouracil (3.0 g, 12.6 mmol) and dry DMF (10 ml). Sodium hydride (362 mg, 15.1 mmol) was added portion wise at −5° C. and stirred for 15 min at 0° C. 3-Cyanobenzyl bromide (2.9 g, 15.1 mmol) was added drop wise at 0° C. and the reaction mixture was stirred for 2 h at room temperature. It was then poured into ice water (25 ml) and extracted with ethyl acetate

(3×30 ml). The combined organic layer was washed with brine, dried over sodium sulfate and concentrated under vacuum. The crude product was purified by column chromatography using 2:8 ethyl acetate:dichloromethane. Yield=1.2 g (27%)

The proton NMR data of the desired product, 1-benzyl-5-iodo-1H-pyrimidine-2,4-dione, is provided below:

¹H NMR (400 MHz, DMSO-d₆, δppm):

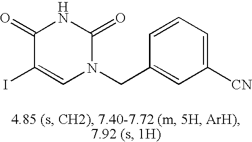

### B. Preparation of 3-(5-furan-2-yl-2,4-dioxo-3,4-dihydro-2H-pyrimidin-1-ylmethyl)-benzonitrile

A 25 ml single neck round bottom flask was charged with 1-benzyl-5-iodo-1H-pyrimidine-2,4-dione (500 mg, 1.41 mmol), tributyl-furan-2-yl-stannane (606 mg, 1.69 mmol), PdCl₂(PPh₃)₂ (50 mg, 0.69 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=100 mg (24%)

The proton NMR data of the desired product, 3-(5-furan-2-yl-2,4-dioxo-3,4-dihydro-2H-pyrimidin-1-ylmethyl)-benzonitrile, is provided below:

¹H NMR (400 MHz, DMSO-d₆, δppm):

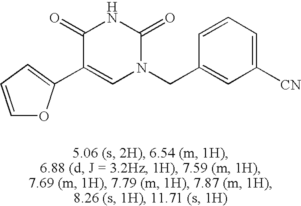

### EXAMPLE 19

### 4-(2,4-dioxo-5-thiophen-2-yl-3,4-dihydro-2H-pyrimidin-1-ylmethyl)-benzonitrile

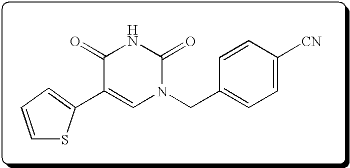

### A. Preparation of 5-iodo-1-(4-cyano-benzyl)-1H-pyrimidine-2,4-dione

A 25 ml two neck round bottom flask was charged with 5-iodouracil (1.5 g, 6.3 mmol) and dry DMF (10 ml). Sodium hydride (181 mg, 7.56 mmol) was added portion wise at −5° C. and stirred for 15 min at 0° C. 4-Cyanobenzyl bromide (1.5 g, 7.56 mmol) was added at 0° C. and the reaction mixture was stirred for 2 h at room temperature. It was then poured into ice water (25 ml) and extracted with ethyl acetate (3×30 ml). The combined organic layer was washed with brine, dried over sodium sulfate and concentrated under vacuum. The crude product was purified by column chromatography using 2:8 ethyl acetate:dichloromethane. Yield=230 mg (10.3%)

The proton NMR data of the desired product, 5-iodo-1-(4-cyano-benzyl)-1H-pyrimidine-2,4-dione, is provided below:

¹H NMR (400 MHz, DMSO-d₆, δppm):

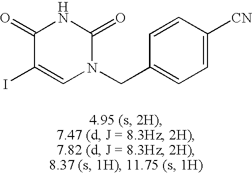

### B. Preparation of 4-(2,4-dioxo-5-thiophen-2-yl-3,4-dihydro-2H-pyrimidin-1-ylmethyl)-benzonitrile

A 25 ml single neck round bottom flask was charged with 5-iodo-1-(4-cyano-benzyl)-1H-pyrimidine-2,4-dione (500 mg, 1.47 mmol), tributyl-thiophene-2-yl-stannane (629 mg, 1.76 mmol), PdCl₂(PPh₃)₂ (50 mg, 0.07 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=40 mg (9%)

The proton NMR data of the desired product, 4-(2,4-dioxo-5-thiophen-2-yl-3,4-dihydro-2H-pyrimidin-1-ylmethyl)-benzonitrile, is provided below:

¹H NMR (400 MHz, DMSO-d₆, δppm):

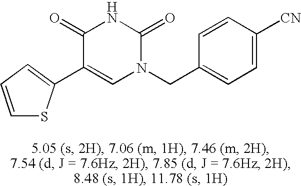

### EXAMPLE 20

### 3-(3,5-difluoro-benzyl)-5-furan-2-yl-1H-pyrimidine-2,4-dione

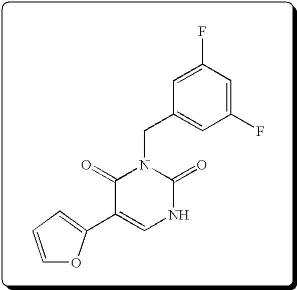

### A. Preparation of 5-iodo-2,4-dioxo-3,4-dihydro-2H-pyrimidin-1-carboxylic Acid Tert-Butyl Ester

5-Iodo-2,4-dioxo-3,4-dihydro-2H-pyrimidin-1-carboxylic acid tert-butyl ester was prepared by adapting the method as described in Example 8.

### B. Preparation of 3-(3,5-difluoro-benzyl)-5-iodo-1H-pyrimidine-2,4-dione

A 25 ml two neck round bottom flask was charged with 5-iodo-2,4-dioxo-3,4-dihydro-2H-pyrimidin-1-carboxylic acid tert-butyl ester (2.0 g, 5.9 mmol) and dry DMF (10 ml). Sodium hydride (169 mg, 7.0 mmol) was added portion wise at −5° C. and stirred for 15 min at 0° C. 3,5-Difluorobenzyl bromide (1.46 g, 7.0 mmol) was added at 0° C. and the reaction mixture was stirred for 3 h at room temperature. It was then poured into ice water (25 ml) and extracted with ethyl acetate (3×30 ml). The combined organic layer was washed with brine, dried over sodium sulfate and concentrated under vacuum. The crude product was dissolved in methanol (10 ml) and potassium carbonate (820 mg, 5.9 mmol) was added and stirred for 2 h at room temperature. Methanol was removed under reduced pressure and water (5 ml) was added and extracted with ethyl acetate (3×10 ml). The combined organic layer was washed with brine, dried over sodium sulfate and concentrated under vacuum. The crude product was purified by column chromatography using ethyl acetate:hexane in the ratio of 20:80. Yield=800 mg (38%). The proton NMR data of the desired product, 3-(3,5-difluoro-benzyl)-5-iodo-1H-pyrimidine-2,4-dione, is provided below: ¹H NMR (400 MHz, DMSO-d₆, δppm):

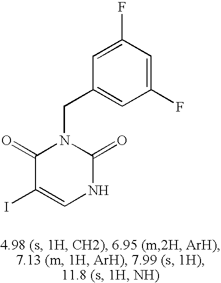

### C. Preparation of 3-(3,5-difluoro-benzyl)-5-furan-2-yl-1H-pyrimidine-2,4-dione

A 25 ml single neck round bottom flask was charged with 3-(3,5-difluoro-benzyl)-5-iodo-1H-pyrimidine-2,4-dione (500 mg, 1.37 mmol), tributyl-furan-2-yl-stannane (588 mg, 1.6 mmol), PdCl₂(PPh₃)₂ (48 mg, 0.06 mmol) and dry DMF (5 ml). The reaction mixture was refluxed for 2 h at 170° C. It was then cooled to room temperature, diluted with ethyl acetate (20 ml) and washed with water (10 ml). The organic layer was stirred with saturated KF solution for 12 h, separated and washed with water (2×10 ml) followed by brine (10 ml), dried over sodium sulfate and concentrated under vacuum. The crude product was purified by column chromatography using ethyl acetate:hexane in the ratio of 20:80. Yield=150 mg (53.5%) The proton NMR data of the desired product, 3-(3,5-difluoro-benzyl)-5-furan-2-yl-1H-pyrimidine-2,4-dione, is provided below:

¹H NMR (400 MHz, DMSO-d₆, δppm):

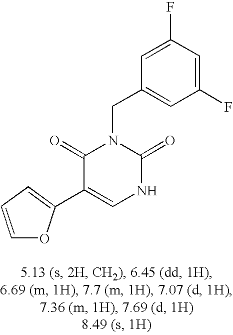

We claim:

1. A compound of formula (I) 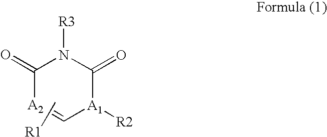
or its stereoisomers, tautomers, solvates, hydrates, pharmaceutically acceptable salts or mixtures thereof, wherein A₁ is Nitrogen; A₂ is Carbon;
R1 is selected from optionally substituted aryl, 5-6 membered heterocyclyl and heteroaryl with 1-3 hetero atoms independently selected from N, O, and S, wherein the substituents are selected from a group consisting of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said 5-6 membered heterocyclyl and heteroaryl exclude nucleosides, sugar moieties, oligomers, peptides, and polymeric ring systems;
R2 and R3 are independently selected from H, alkyl, —(CH₂)ₙ—Y wherein n=0 to 4, and Y is selected from a group consisting of alkyne, alkoxy, ester, carbonyl, carboxyl, aryl, cycloalkyl, heteroaryl and heterocyclyl which are further optionally substituted with halogen, cyano, nitro, alkyl, hydroxyl, alkoxy, carboxyl groups; with the proviso that either R2 or R3 is H and R2 is not same as R3.

2. A compound as claimed in claim 1 or its stereoisomers, tautomers, solvates, hydrates, pharmaceutically acceptable salts or mixtures thereof, wherein A₁ is Nitrogen; A₂ is Carbon;
R1 is selected from optionally substituted aryl, 5-6 membered heterocyclyl and heteroaryl with 1-3 hetero atoms independently selected from N, O, and S, wherein the substituents are selected from a group comprising of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said 5-6 membered heterocyclyl and heteroaryl exclude nucleosides, sugar moieties, oligomers, peptides, and polymeric ring systems;
R2 and R3 are independently selected from H, alkyl, —(CH₂)ₙ—Y wherein n=0 to 4, and Y is selected from a group consisting of ester, aryl, which are further optionally substituted with 1-5 halogen, cyano, nitro, alkyl, hydroxyl, alkoxy, carbonyl, carboxyl groups; with the proviso that either R2 or R3 is H and R2 is not same as R3.

3. A compound as claimed in claim 1 or its stereoisomers, tautomers, solvates, hydrates, pharmaceutically acceptable salts or mixtures thereof, wherein A₁ is Nitrogen; A₂ is Carbon;
R1 is selected from optionally substituted heteroaryl with 1-3 hetero atoms independently selected from N, O, and S, wherein the substituents are selected from a group comprising of H, halogen, substituted or unsubstituted lower alkyl, alkyne, alkenes, cycloalkyl, alkoxy, esters, carbonyl, carboxyl, alcohols, substituted or unsubstituted aryl, substituted or unsubstituted heteroaryl; with the proviso that the said heteroaryl excludes nucleosides, sugar moieties, oligomers, peptides, and polymeric ring systems;
R2 and R3 are independently selected from H, alkyl, —(CH₂)ₙ—Y wherein n=0 to 4, and Y is selected from a group consisting of ester, aryl, which are further optionally substituted with 1-5 halogen, cyano, nitro, alkyl, hydroxyl, alkoxy, carboxyl groups; with the proviso that either R2 or R3 is H and R2 is not same as R3.

4. A compound as claimed in claim 1 or its stereoisomers, tautomers, solvates, hydrates, pharmaceutically acceptable salts or mixtures thereof, wherein A₁ is Nitrogen; A₂ is Carbon;
R1 is a heteroaryl wherein the heteroatom is selected from N, O, and S; with the proviso that the said heteroaryl excludes nucleosides, sugar moieties, oligomers, peptides, and polymeric ring systems;
R2 and R3 are independently selected from H, (CH₂)ₙ—Y wherein n=1 to 4 and Y is selected from a group consisting of ester, aryl, which are further optionally substituted with 1-2 halogen, cyano, nitro, alkyl, hydroxyl, alkoxy, carboxyl groups; with the proviso that either R2 or R3 is H and R2 is not same as R3.

5. The compound as claimed in claim 1, wherein said compound is selected from the group consisting of 3-benzyl-5-thiophen-2-yl-1H-pyrimidine-2,4-dione; and 3-(5-thiophen-2-yl-2,6-dioxo-3,6-dihydro-2H-pyrimidin-1-yl-methyl)benzonitrile or a pharmaceutically acceptable salt thereof.

6. The compound as claimed in claim 1, wherein said compound is selected from the group consisting of ethyl ester of (2,4-dioxo-5-thiophen-2-yl-3,4-dihydro-2H-pyrimidin-1-yl)acetic acid; 3-(4-nitrobenzyl)-5-furan-2-yl-1H-pyrimidine-2,4-dione; 3-benzyl-5-furan-2-yl-1H-pyrimidine-2,4-dione; 3-(5-furan-2-yl-2,6-dioxo-3,6-dihydro-2H-pyrimidin-1yl-methyl)benzonitrile; 1-(4-fluorobenzyl)-5-thiophen-2-yl-1H-pyrimidine-2,4-dione; and 3-(4-nitrobenzyl)-5-thiophen-2-yl-1H-pyrimidine-2,4-dione or a pharmaceutically acceptable salt thereof.

7. The compound as claimed in claim 1, wherein said compound is selected from the group consisting of 1-(4-fluorobenzyl)-5-furan-2-yl-1H-pyrimidine-2,4-dione; 3-(3,5-difluorobenzyl)-5-furan-2-yl-1H-pyrimidine-2,4-dione; 4-(5-furan-2-yl-2,6-dioxo-3,6-dihydro-2H-pyrimidin-1yl-methyl)benzonitrile; 3-(4-fluorobenzyl)-5-furan-2-yl-1H-pyrimidine-2,4-dione; 3-(5-thiophen-2-yl-2,6-dioxo-3,6-dihydro-2H-pyrimidin-1-yl-methyl)benzonitrile; and 1-benzyl-5-thiophen-2-yl-1H-pyrimidine-2,4-dione or a pharmaceutically acceptable salt thereof.

8. The compound as claimed in claim 1, wherein said compound is selected from the group consisting of 3-(5-furan-2-yl-2,6-dioxo-3,6-dihydro-2H-pyrimidin-1-yl-methyl)benzonitrile; ethyl ester of (2,6-dioxo-5-furan-2-yl-3,6-dihydro-2H-pyrimidin-1-yl)acetic acid; 4-(5-thiophen-2-yl-2,4-dioxo-3,4-dihydro-2H-pyrimidin-1-yl-methyl)benzonitrile; 4-(5-furan-2-yl-2,6-dioxo-3,6-dihydro-2H-pyrimidin-1-yl-methyl)benzonitrile; ethyl ester of (2,4-dioxo-5-furan-2-yl-3,4-dihydro-2H-pyrimidin-1-yl)acetic acid; and 3-(4-fluorobenzyl)-5-thiophen-2-yl-1H-pyrimidine-2,4-dione or a pharmaceutically acceptable salt thereof.

9. A pharmaceutical composition comprising a compound of claim 1 or a pharmaceutically acceptable salt thereof, in combination with a pharmaceutically acceptable carrier or diluents.

10. A pharmaceutical composition for use in the prophylaxis or treatment of conditions associated with protein kinase inhibition, comprising, as active ingredient, a pharmaceutically effective amount of the compound of claim 1 in association with pharmaceutically acceptable carriers or diluents.

11. A method for the prophylaxis or treatment of a medical condition associated with protein kinase, the method comprising administering to a patient in need of such prophylaxis or treatment a pharmaceutically effective amount of the compound of claim 1 or salt thereof.

12. A method for the prophylaxis or treatment of a medical condition associated with protein kinase as claimed in claim 10, wherein the medical condition can be of dementia, Alzheimer's Disease, Parkinson's Disease, Frontotemporal dementia Parkinson's Type, Parkinson dementia complex of Gaum, HIV dementia, diseases with associated neurofibrillar tangle pathologies, amyotrophic lateral sclerosis, corticobasal degeneration, dementia pugilistica, Down syndrome, Huntington's Disease, postencephalitic parkinsonism, progressive supranuclear palsy, Niemann-Pick's Disease, Pick's Disease, stroke, head trauma and other chronic neurodegenerative diseases, Bipolar Disease, affective disorders, depression, schizophrenia, cognitive disorders, Type I and Type II diabetes, diabetic neuropathy and hair loss.
